(19) United States
(12) Patent Application Publication
(10) Pub. No.: US 2026/0265665 A1
(43) Pub. Date: Sep. 10, 2026

(54) REACTOR AND METHOD OF USING THE SAME

(71) Applicant: ASKLEPIOS BIOPHARMACEUTICAL, INC., Research Triangle Park, NC (US)

(72) Inventors: Alfredo Martinez MOGARRA, Research Triangle Park, NC (US); Sergio Martin PARRÓN, Research Triangle Park, NC (US); Felix D. BASTIDA-CORCUERA, Research Triangle Park, NC (US)

(21) Appl. No.: 18/871,921

(22) PCT Filed: Jun. 13, 2023
(86) PCT No.: PCT/US2023/025190

§ 371 Date: Dec. 5, 2024

(30) Foreign Application Priority Data
Jun. 14, 2022 (EP) 22382567.0
Aug. 24, 2022 (EP) 22191879.0

(51) Int. Cl. C12M 1/34; C12M 1/00; C12M 1/12; C12M 3/06; C12P 19/34

(52) U.S. Cl. CPC C12M 41/12; C12M 23/04; C12M 23/20; C12M 23/22; C12M 23/38; C12M 27/16; C12P 19/34

(57) ABSTRACT

A reactor includes a support base, at least one wall, a top cover and a moveable plate assembly. The top cover, the support base and the at least one wall form an internal chamber therein. The moveable plate assembly includes a moveable plate located within the internal chamber. The moveable plate being configured to move in the x-direction, y-direction, and the z-direction. The temperature of the moveable plate is controllable to a desired temperature. The internal chamber is accessible via at least one of the top cover, the support base, and the at least one wall.

### CROSS-REFERENCE TO RELATED APPLICATIONS

This application claims priority to European Patent Application No. 22382567.0 filed on Jun. 14, 2022, and European Patent Application No. 22191879.0 filed on Aug. 24, 2022, each of which is hereby incorporated by reference herein in its entirety.

### FIELD OF THE INVENTION

This application is directed to reactors or bioreactors and, more specifically, is directed to reactors to be used in reactions that require temperature control and/or controlled homogenous mixing.

### BACKGROUND OF THE INVENTION

Reactors or bioreactors have been used in the past in enzymatic reactions. These reactors are used in cell culture (eukaryotic or prokaryotic cells). These reactors are not user friendly for larger volumes, and often also involve steps such as manually shaking the bag containing the material from time to time. Manual steps such as shaking are often disadvantageous in reactions, especially those reactions involving larger strands that may be prone to potentially nicks that will break the longer chains. If the material is viscous and shook with improper mechanical forces, the chains can break down, resulting in reduced production yields. This can happen frequently with, for example, DNA. It would be desirable to have a reactor that addresses these disadvantages of existing reactors, while still being of a desired industrial scale and flexibility in the manufacturing methods.

### SUMMARY

According to one aspect of the present disclosure, a reactor comprises a support base, at least one wall, and a top cover. The top cover, the support base and the at least one wall form an internal chamber therein. The reactor further comprises a moveable plate assembly including a moveable plate located within the internal chamber. The moveable plate is configured to move in the x-direction, y-direction, and the z-direction. The temperature of the moveable plate is controllable to a desired temperature. The internal chamber is accessible via at least one of the top cover, the support base, and the at least one wall.

According to a configuration of the above implementation, the top cover, the support base, and the at least one wall comprise stainless steel.

According to a configuration of the above implementation, the at least one wall forms a porthole for viewing into the internal chamber. In one implementation, the at least one wall is a plurality of walls and the plurality of walls forms a respective porthole for viewing into the internal chamber.

According to another configuration of the above implementation, the internal chamber is accessible by moving a portion of the at least one wall and a portion of the top cover via a hinge.

According to a further configuration of the above implementation, the moveable plate comprises aluminum.

In a further aspect of the above implementation, the moveable plate comprises a plurality of layers. In one implementation, the moveable plate comprises a resistive heating element layer.

In a further aspect of the above implementation, the maximum inclination of the moveable plate is about 20 degrees or about 30 degrees in any direction.

In yet a further aspect of the above implementation, the movement of the moveable plate uses linear movement, rotary movement and orbital movement.

According to another aspect of the present disclosure, the reactor has a width of from about 500 mm to about 1,500 mm, a height of from about 500 mm to about 2,000 mm, and a depth of from about 500 mm to about 1,500 mm.

According to a configuration of the above implementation, filters are further included to assist in preventing or inhibiting cross-contamination.

According to another configuration of the above implementation, the at least one wall is a plurality of walls.

According to a further configuration of the above implementation, the internal chamber is hermetically closed.

According to another aspect of the present disclosure, a reactor is provided that comprises a support base, at least one wall, and a top cover. The top cover, the support base and the at least one wall form an internal chamber therein. The reactor further comprises a moveable plate assembly including a moveable plate located within the internal chamber. The moveable plate is configured to move in the x-direction, y-direction, and the z-direction. The temperature of the moveable plate is controllable to a desired temperature. The internal chamber is accessible via at least one of the top cover, the support base, and the at least one wall. A bag or package is placed on the moveable plate in a secured position. The bag includes at least one reaction component and an enzyme. The moveable plate is heated to a desired temperature. The temperature is controlled within the internal chamber. The moveable plate is moveable in the x-direction, y-direction and the z-direction so as to mix the at least one reaction component and the enzyme.

According to a configuration of the above implementation, the bag is from about 1 to about 20 liters.

According to a configuration of the above implementation, the at least one reaction component is a nucleic acid. The nucleic acid may be DNA and/or RNA.

According to another configuration of the above implementation, the at least one reaction component and an enzyme involves restriction digestion with restriction endonucleases; nucleic acid ligation; reactions with telomerase or protelomerase; reactions with endonuclease and/or exonuclease; reactions with DNA polymerase or any variant thereof; reactions with reverse transcriptase; reactions with recombinase; reaction with protease; reaction with nuclease; polymerase chain reaction; nucleic acid amplification; phosphorylation reaction; dephosphorylation reaction; nucleic acid synthesis reaction; reaction with RNA polymerase; nick or damage repair reaction of nucleic acid; nucleic acid denaturation reaction; nucleic acid annealing reaction; nucleic acid extension reaction; or any combination thereof.

According to a further configuration of the implementation, the at least one reaction component and an enzyme involves restriction endonucleases; nucleic acid; amino acid; ligase; telomerase or protelomerase; deoxynucleoside triphosphate (dNTPs); endonuclease and/or exonuclease; DNA polymerase or any variant thereof; reverse transcriptase; recombinase, protease; nuclease; Taq DNA polymerase; phosphorylase; phosphotransferase; dephosphorylase; nucleic acid synthase; RNA polymerase; or any combination thereof.

According to another embodiment, the at least one reaction component is RNA, DNA, plasmid DNA, deoxynucleoside triphosphate (dNTPs), oligonucleotides, inhibitory nucleic acids, gRNA, modified DNA, or any combination thereof.

According to another embodiment, the enzyme is involved in an enzymatic reaction. The enzymatic reaction is restriction digestion with restriction endonucleases; nucleic acid ligation; reaction with protelomerase; reaction with endonuclease and/or exonuclease; reaction with DNA polymerase or any variant thereof; reaction with reverse transcriptase; reaction with recombinase; reaction with protease; reaction with nuclease; polymerase chain reaction; nucleic acid amplification reaction; phosphorylation reaction; dephosphorylation reaction; nucleic acid synthesis reaction; reaction with RNA polymerase; reaction of nick or damage repair of nucleic acid; nucleic acid denaturation reaction; nucleic acid annealing reaction; or nucleic acid extension reaction.

The above summary is not intended to represent each embodiment or every aspect of the present invention. Additional features and benefits of the present invention are apparent from the detailed description and figures set forth below.

### BRIEF DESCRIPTION OF DRAWINGS

Other advantages of the invention will become apparent upon reading the following detailed description and upon reference to the drawings in which:

FIG. 1 is a generally front and side perspective view of a reactor or a bioreactor according to one embodiment.

FIG. 2A is a top perspective view of a moveable plate according to one embodiment that is used in the reactor of FIG. 1.

FIG. 2B is an enlarged view of the moveable plate used in FIG. 2A.

FIG. 2C is a moveable plate according to another embodiment.

FIG. 3 is a top perspective view of a resistive heating element layer used in the moveable plate of FIGS. 2A, 2B according to one embodiment.

FIG. 4A is a bottom perspective view of a moveable plate assembly, including the moveable plate of FIG. 2A, in one position.

FIG. 4B is a side perspective view of the moveable plate assembly of FIG. 4A in another position.

FIG. 5A is a top perspective view of a moveable plate assembly including an adjustable locking system according to one embodiment.

FIG. 5B is a top perspective view of a moveable plate assembly including an adjustable locking system according to another embodiment.

FIG. 6 is a back perspective view of a mechanical movement system to move the moveable plate assembly of FIGS. 4A and 4B according to one embodiment.

FIG. 7A is a side view of a reactor according to another embodiment.

FIG. 7B is a front view of the reactor of FIG. 7A.

FIG. 7C is a partial side perspective view of the reactor of FIG. 7A.

FIG. 8 is an exploded view of a flow sensor to measure viscosity according to one embodiment.

FIG. 9A is a bag or package that is used with a reactor according to one embodiment.

FIG. 9B is a bag or package secured to the moveable plate assembly of FIG. 5B according to another embodiment.

FIG. 10 is a graph of the temperature data of 445 ml of water being heated from room temperature to 30° C.

FIG. 11 is a graph of the temperature data of 4450 ml of water being heated from room temperature to 30° C.

FIG. 12 is a graph of the temperature data of 4450 ml of water being heated and cooled between 3° and 50° C.

While the invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

This application in one embodiment is directed to a reactor. The reactor includes a support base, at least one wall, a top cover, and a moveable plate assembly. The top cover, the support base, and the at least one wall form an internal chamber therein that is hermetically closed. The moveable plate assembly includes a moveable plate located within the internal chamber. The moveable plate is configured to move in the x-direction, y-direction, and the z-direction. The temperature of the moveable plate is controllable to a desired temperature. The internal chamber of the reactor is accessible via at least one of the top cover, the support base, and the at least one wall. In one embodiment, the reactor is an incubation system capable of mixing materials (e.g., genetic material) and maintaining it at a desired or required temperature.

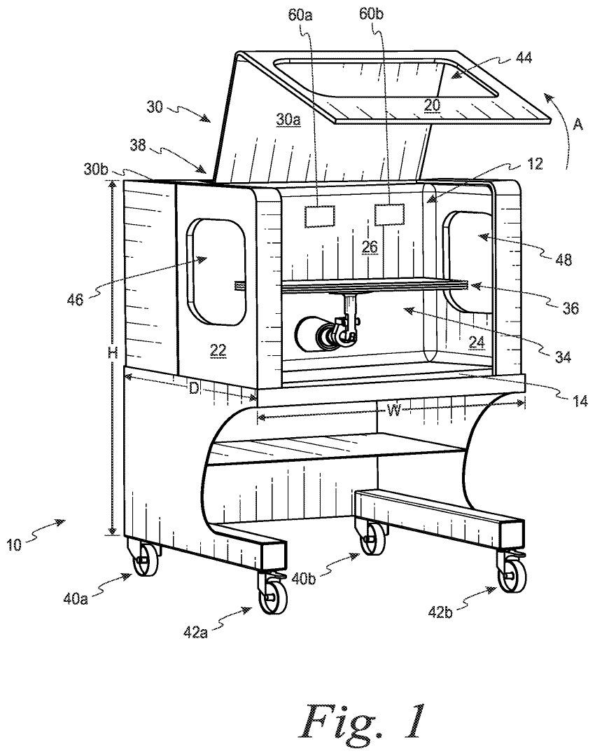

Referring to FIG. 1, a reactor or bioreactor 10 is shown according to one embodiment. The reactor 10 is in an open position such that a user can access an internal chamber 12 of the reactor 10. The reactor 10 includes a support base 14, a plurality of walls 20, 22, 24, 26, a top cover 30, and a moveable plate assembly 34. The combination of the plurality of walls 20, 22, 24, 26, the top cover 30, and the support base 14 form the internal chamber 12 therein, which is hermetically closed and sealed in this embodiment. Thus, the reactor is a fully isolated apparatus from the surrounding environment in one embodiment. The reactor 10 has a depth D, a width W and a height H.

The internal chamber 12 of FIG. 1 is accessible by moving the top cover 30 and the front wall 20. In this embodiment, the top cover 20 and the front wall 30 are integrally formed. More specifically, the internal chamber 12 is accessible by moving or pivoting the front wall 20 and a first portion 30a of the top cover 30 via a hinge 38 along the general path of arrow A to an open position. This type of opening of the reactor to access the internal chamber 12 may be referred to as an ascending opening. A second portion 30b of the top cover 30 remains stationary when the internal chamber 12 is being accessed. It is contemplated that an internal chamber of a reactor may be accessed by other methods. For example, accessing of the internal chamber may involve movement of one or more of the front wall, sidewalls, back wall and/or top cover.

The shape of the internal chamber 12 of the reactor 10 in FIG. 1 is generally rectangular. It is contemplated that an internal chamber of a reactor may be other polygonal shapes such as square, a pentagon, a hexagon or octagon. It is also contemplated that the shape of an internal chamber of a reactor may be non-polygonal such as generally oval or circular. It is contemplated that the shape of the internal chamber may be of other polygonal and non-polygonal shapes than specifically mentioned above.

The reactor 10 as shown in FIG. 1 includes a plurality of wheels 40a, 40b, 42a, 42b at a bottom thereof. The wheels 40a, 40b are in a fixed position, while the wheels 42a, 42b are mobile wheels. The plurality of wheels 40a, 40b, 42a, 42b assists in easily moving the reactor 10. It is contemplated that a reactor may be designed with different wheels or no wheels at all.

At least one of the plurality of walls may form a porthole for viewing into the internal chamber. Referring still to FIG. 1, the front wall 20 forms a porthole 44, the side wall 22 forms a porthole 46, and the side wall 24 forms a porthole 48. The portholes 44, 46, 48 may be double-glazed portholes with ultraviolet (UV) protection. In such an embodiment, the portholes 44, 46, 48 allow desirable visibility of the process occurring in the internal chamber 12 of the reactor 10. It is contemplated that the walls of a reactor may include more or less portholes than shown in FIG. 1, including no portholes.

The moveable plate assembly 34 includes a moveable plate 36. The moveable plate 36 is located within the internal chamber 12 of the reactor 10 of FIG. 1. The moveable plate 36 includes a plurality of layers in this embodiment. It is contemplated that a moveable plate may be made of a single layer in another embodiment.

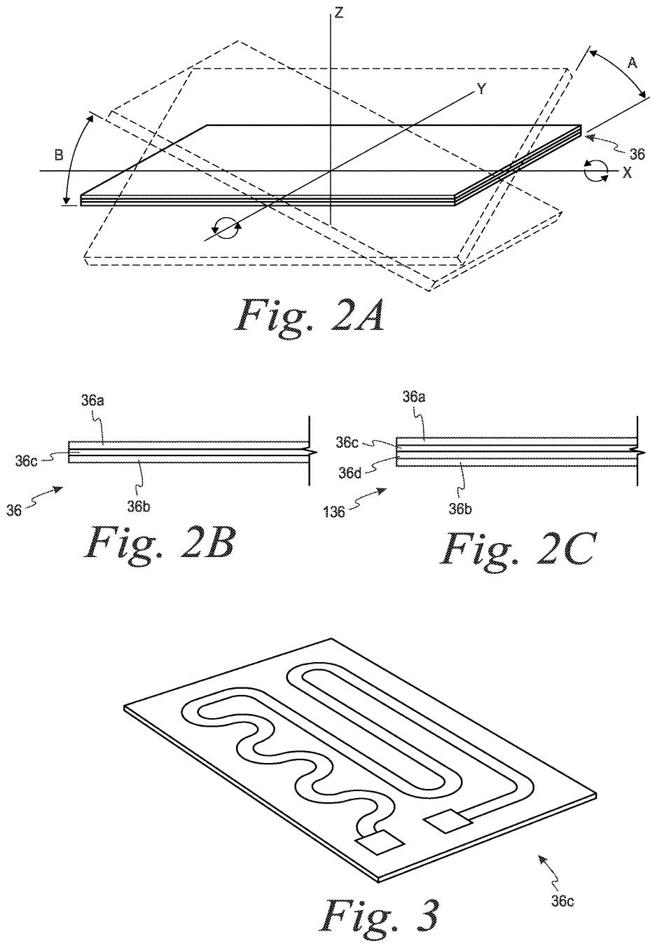

FIG. 2A shows a top perspective view of a moveable plate 36 with potential positions or locations, while FIG. 2B is an enlarged view of a portion of the moveable plate 36 of FIG. 2A showing its individual layers. As shown best in FIG. 2A, the moveable plate 36 is configured to move in the x-direction, y-direction, and the z-direction. The maximum inclination of the moveable plate 36 is shown as angles A, B in FIG. 2A. In one embodiment, the maximum inclination of the moveable plate 36 is about 20 degrees in any direction. In another embodiment, the maximum inclination of the moveable plate 36 is about 25 degrees in any direction. In a further embodiment, the maximum inclination of the moveable plate 36 is about 30 degrees in any direction. The inclinations of the moveable plate 36 is generally from about 5 to about 25 degrees in any direction. In other embodiments, the inclinations of the moveable plate are from about 10 to about 25, or from about 10 to 20 degrees in any direction.

It is contemplated that the maximum inclinations can vary between the x-direction, y-direction, and the z-direction. It is also contemplated that the maximum inclinations can be the same in the x-direction, y-direction, and the z-direction.

FIG. 2B shows the moveable plate 36 including a first moveable plate layer 36a, a second moveable plate layer 36b, and a resistive heating element layer 36c. The resistive heating element layer 36c is located between the first and second moveable plate layers 36a, 36b.

It is also contemplated that additional layers may be included in the moveable plate such as a silicon layer. If a silicone layer is used, it is typically directly adjacent to the resistive heating element layer 36c. One non-limiting example is shown in FIG. 2C with a moveable plate 136. The moveable plate 136 includes the first moveable plate layer 36a, the second moveable plate layer 36b, the resistive heating element layer 36c and a silicone layer 36d. The resistive heating element layer 36c is located between the first moveable plate layer 36a and the silicone layer 36d. The silicone layer 36d is located between the resistive heating element layer 36c and the second moveable plate layer 36b. It is also contemplated that additional layers may be included in the moveable plate.

The first and second moveable plate layers 36a, 36b in one embodiment comprise aluminum. The aluminum may be treated such as with an anodic oxidation treatment. Aluminum is especially desirable because of its properties includes heat transfer. It is contemplated that the first and second moveable plate layers may comprise other materials such as steel.

Referring to FIGS. 2B, 2C and 3, the resistive heating element layer 36c is configured to heat the moveable plate 36. The resistive heating element layer 36c has electrical current that passes therethrough that generates heat due to the resistive nature of the element's design. Resistive heating element layers may comprise metallic alloys, ceramic materials, or ceramic metals.

The temperature of the moveable plate 36 is controllable to a desired temperature in this embodiment. In one embodiment, the temperature of the moveable plate 36 heated by the resistive heating element layer 36c is controlled by a plurality of calibrated probes (not shown). One non-limiting example of a calibrated probe that may be used to control the temperature of the moveable plate is a PT-100 probe. One probe is typically used for a smaller bag or package, while two probes are typically used for larger bags or packages. It is contemplated that other probes and methods of controlling the temperature of the moveable plate may be used. It is also contemplated that a further probe can be used with an upper temperature limit that can be configured to send an alarm and stop the reactor when the temperature has exceeded this upper temperature limit.

The moveable plate 36 in one embodiment is adapted to be heated up to about 100° C. The moveable plate 36 in another embodiment is adapted to be heated up to about 120° C. The moveable plate 36 in a further embodiment is adapted to be heated up to about 135° C.

The moveable plate 36 can vary in terms of duration to heat up to a desired temperature. The moveable plate in one process is set at a desired temperature to be heated to. The rate of heating of the movable plate can vary but is generally from about 0.01° C./min to about 0.30° C./min, and more specifically, from about 0.05° C./min to about 0.20° C./min. In another rate of heating, the movable plate is from about 0.05° C./min to about 0.15° C./min, and more specifically, from about 0.10° C./min to about 0.15° C./min. It is contemplated that other heating rates may be run. During heating of the moveable plate, the reactor may ventilate air in a recirculating or external manner. Ventilating of the moveable plate during heating is typically done in a recirculating manner.

In another embodiment, the moveable plate may have a cooling plate layer instead of or in addition to a resistive heating element layer. In such an embodiment, the moveable plate would be cooled instead of heated. If both cooling and heating is desired, water heat exchangers may be used in one embodiment. Hot water can be produced with either a heat pump or resistive heating element (for hot water), which cold water can be produced by Peltier cooler. In this embodiment, the temperature of the moveable plate may be controlled by similar techniques as described above.

The moveable plate 36 can vary in terms of duration to cool down to a desired temperature. The moveable plate in one process is set at temperature to be cooled to. The rate of heating of the movable plate can vary but is generally from about 0.005° C./min to about 0.15° C./min, and more specifically, from about 0.01° C./min to about 0.10° C./min. In another rate of heating, the movable plate is from about 0.05° C./min to about 0.15° C./min, and more specifically, from about 0.10° C./min to about 0.15° C./min. It is contemplated that other cooling rates may be run. During cooling, the reactor may ventilate air in a recirculating or external manner. Ventilating of the moveable plate during cooling is typically done in an external manner.

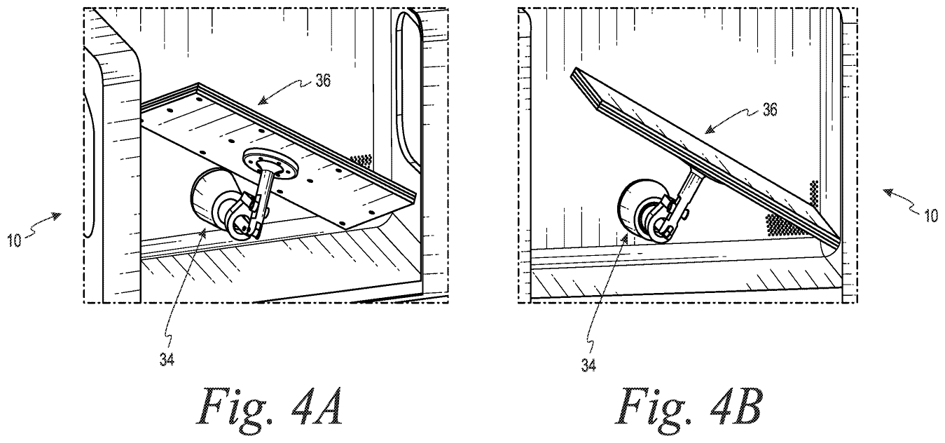

FIGS. 4A, 4B show the moveable plate assembly 34 including the moveable plate 36. FIG. 4A shows the moveable plate 36 in one position within the reactor 10, while FIG. 4B shows the moveable plate 36 in another position within the reactor.

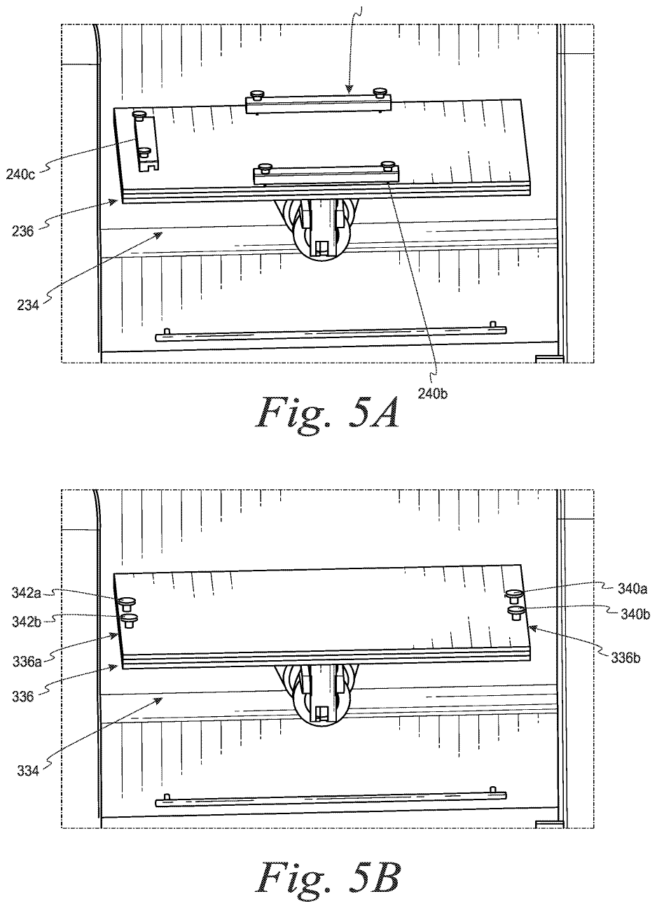

FIG. 5A is a top perspective view of a moveable plate assembly 234 that includes a moveable plate 236 including an adjustable locking system according to one embodiment. The moveable plate 236 is the same as the moveable plate 36 described in detail above, except for the added adjustable clamps 240a, 240b, and 240c. It is contemplated that the adjustable locking system may also be an adjustable locking and sealing system.

The adjustable clamps 240a, 240b, and 240c secure or hold a bag or package on the moveable plate thereon in one embodiment. The adjustable clamps 240a, 240b, and 240c are designed and positioned to work with bag or packages of different sizes. The adjustable clamps 240a, 240b, and 240c are located generally on three sides of the moveable plate 236. More specifically, the adjustable clamps 240a, 240b, and 240c are located near or at the periphery of the moveable plate 236. As shown in FIG. 5A, an adjustable clamp is not typically located on a side of a bag of package where components are added during the reaction. For example, the bag or package may have a liquid component(s) entering the bag or package via tubes.

It is contemplated that fewer or more adjustable clamps may be used than depicted in FIG. 5A. It is also contemplated that a locking or securing system may be implemented that does not involve adjustable clamps.

For example, FIG. 5B shows a top perspective view of a moveable plate assembly 334 that includes a moveable plate 336 according to one embodiment. The moveable plate 336 is the same as the moveable plate 36 described in detail above, except for the added pairs of knobs 340a, 340b and 342a, 342b. The knobs may be designed to be fixed in one embodiment or may be adjustable in another embodiment. The knobs may also be used in combination with one or more brackets that may be adjustable to secure the bag or package.

The pairs of knobs 340a, 340b and 342a, 342b assist in securing or holding a bag or package on the moveable plate thereon in one embodiment. The pairs of knobs 340a, 340b and 342a, 342b are designed and positioned to work with bag or packages of different sizes, especially if they are adjustable and/or work in combination with one or more brackets. The respective pairs of knobs 340a, 340b and 342a, 342b are located next to each other at opposing ends 336a, 336b of the moveable plate 336. More specifically, the pairs of adjustable knobs 340a, 340b and 342a, 342b are located near or at the periphery of the moveable plate 336.

The moveable plates 36, 136, 236, 336 may be moved by different mechanical components and techniques. The movement of the moveable plate uses both linear movement and rotary movement in one embodiment. The movement of the moveable plate is produced through the interpolarization of linear and rotary movements. In another embodiment, the movement of the moveable plate is produced through the interpolarization of linear, rotary and orbital movements. The oscillations of the moveable plate assembly can vary and are set by a user. The oscillations are generally under about 60 oscillations per minute and, typically, under about 45 oscillations per minute and, more specifically, under 30 oscillations per minute. The oscillations typically range from about 5 to about 50 oscillations per minute, and in more typically are from about 10 to about 40 oscillations per minute. The oscillations are even more typically from about 10 to about 20 oscillations per minute.

The phase delay degrees of the moveable plate may also vary. The phase delay degrees may be 0 degrees, 90 degrees or 180 degrees. In other embodiments, the phase delay degrees can be 30 degrees, 45 degrees, 60 degrees, 75 degrees, 105 degrees, 120 degrees, 135 degrees or 150 degrees. It is contemplated that other phase delay degrees may be used.

The movement of the moveable plate can be setup as a mix of different angles, speeds and phase delays to obtain multiple different shaking modes. For example, the phase delay between both axis x and axis y allows different shakings as orbital, diagonal and mix shakings.

In this embodiment, the movement of the moveable plate is controlled by software including absolute encoders that provide a unique position value at every point of rotation representing the “absolute” position of the encoder. This allows control of the exact position of the rotating shaft in each moment to adjust the inclination and speed selected in moving the moveable plate.

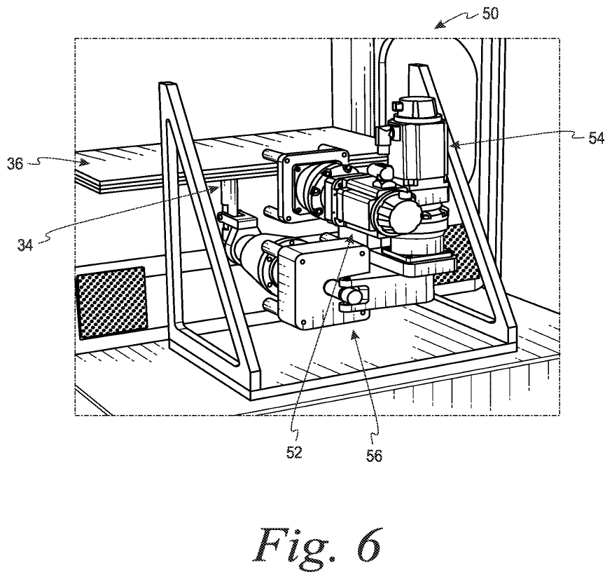

One non-limiting example of a mechanism that can move the moveable plate 36 of the moveable plate assembly 34 is shown in FIG. 6. This mechanism can also move the moveable plates 136, 236, 336 described above.

FIG. 6 shows a mechanical movement system 50 that includes a first brushless motor 52 and a second brushless motor 54. The first and second brushless motors 52, 54 assist in continuously controlling the positions and speed of the moveable plate 36. The first brushless motor 52 is coupled to a pulley and belt system 56 that allows rotary movement. The second brushless motor 54 is coupled to a lever movement that allows linear movement (Y axis inclination). Brushless motors are desirable because of their high power density, good speed-torque characteristics, high efficiency, wide speed ranges, and low maintenance. It is contemplated that other motors may be used to move the moveable plate.

The reactor or bioreactor 10 in one embodiment may further include at least one filter to assist in preventing or inhibiting cross-contamination. A plurality of filters 60a, 60b is shown in FIG. 1 in which the plurality of filters 60a, 60b is mounted on the back wall 26 in the internal chamber 12. A non-limiting example of an air filter that may be used is a HEPA H14 filter. It is contemplated that other filters may be used in the reactor for air filtration.

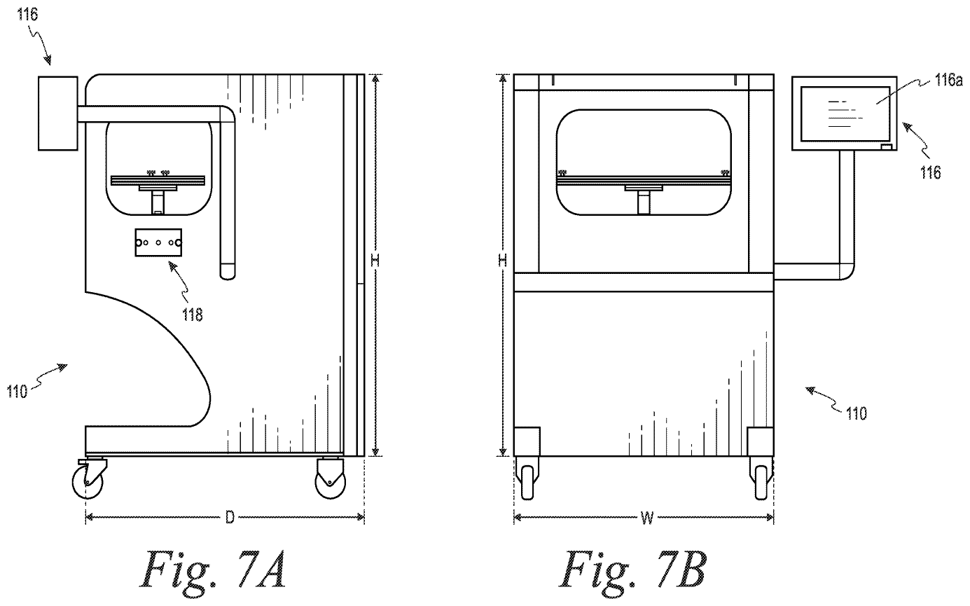

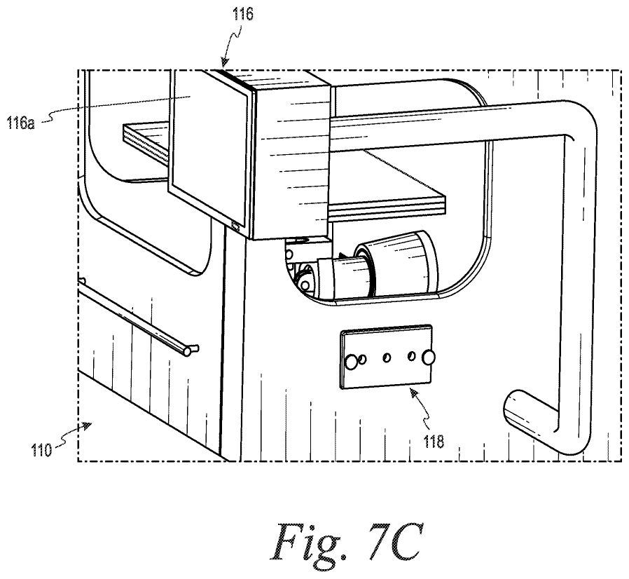

It is contemplated that additional components may be added to the reactor 10. For example, referring to FIGS. 7A-7C, a reactor 110 is shown according to another embodiment. The reactor 110 is substantially the same as the reactor 10 described above, but further includes a monitor 116 with a control panel or screen 116a. The control panel 116a controls the conditions within the reactor 110 including: (1) the temperature conditions of the moveable plate; (2) the temperature of the reactor; and (3) the movement of the moveable plate including the angular movement and the speed of the same. The control panel 116a also controls: (1) the timing of any temperature modifications; and (2) how long and when the movement of the moveable plate assembly occurs. The control panel 116a may control the reactor for a few hours or for several days, if not longer. The control panel 116a may be controlled by touchscreen icons on the screen itself in one embodiment. In another embodiment, the control panel 116a may be controlled by physical buttons or other control mechanisms. It is also contemplated that the control panel in another embodiment may be controlled by a separate computer.

As discussed above, the reactor may be configured to allow liquid to enter the internal chamber during the process. If this is desired, then the reactor may further include a mounted liquid control system with at least one external pump to assist in supplying liquid during the process. The liquid control system allows fluid to exit and re-enter the bag or package located in internal chamber. In one embodiment, the at least one external pump (e.g., a peristaltic pump) transports liquid through flexible tube or hose connections that are connected through a pass-through system (e.g., grommets) in a sidewall. This pass-through system 118 is shown in FIGS. 7A, 7C. One non-limiting example of a peristaltic pump is made by Watson Marlow. This type of pump is desirable because of its ability to be cleaned easily, while still functioning to control the flow of liquid to and from the bag or package. It is contemplated that other pumps may be used.

It is also contemplated that liquid may enter into the bag or package manually during a pause or break in the process steps. In such a process, a user would open the reactor 10 by moving the top cover 30 and the front wall 20 and manually place the liquid into the bag or package.

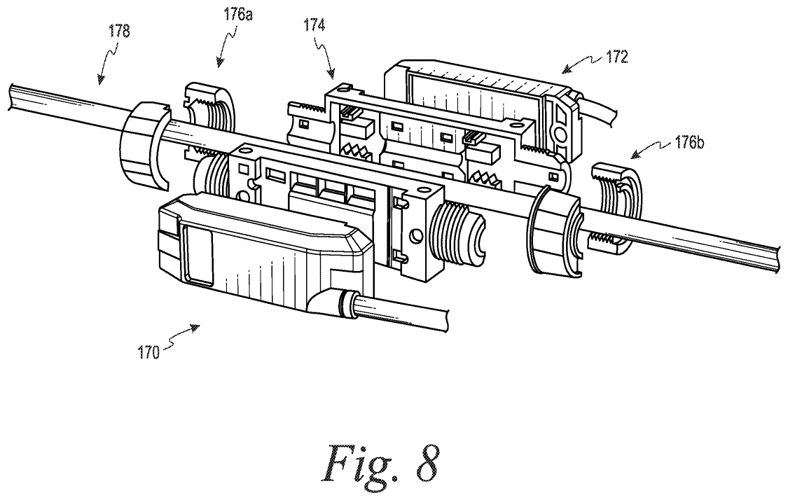

The reactor may further include a viscosity monitoring system to measure and report the viscosity of the components in the bag or package. This may be accomplished by different techniques. For example, a flow sensor may be mounted to a tube where the product itself is made to flow. One non-limiting example of a flow sensor 170 is shown in FIG. 8. The flow sensor 170 includes a sensor head (transceiver) 172, a clamp 174, and a plurality of clamps 176a, 176b. The flow sensor 170 is adapted to be mounted onto a flexible tube 178. One non-limiting commercial example of a flow sensor is the Keyence FD-X envelope flow sensor.

The flow sensor detects the flow velocity in the one or more tubes by crossing them with an ultrasonic beam that will assume different crossing speeds depending on the detected speeds. Here, the velocity will remain constant, but as the viscosity changes, the ultrasound will have a progressively different way of passing through the tubes during the reactor process. The flow sensor does not contact the liquid itself.

In another embodiment, the viscosity changes may be measured by applying Poiseuille law. For example, a peristaltic pump recirculates liquid in the bag or package through a hose or pipe of a certain dimension with a laminar flow (i.e., flow having a Reynolds number (RE)<2300). Two pressure transducers measure the head loss in the hose or pipe. The laminar head loss and viscosity have a linear relationship with each other. This method may be initially calibrated with pure water at 20° C. to measure the viscosity first and then with different carbopol solutions. This method is an inexpensive and fairly accurate method to measure viscosity changes.

The dimensions of the reactor 110 are the same the dimensions of the reactor 10. Specifically, the reactors 10, 110 include the depth D, the width W and the height H. In one embodiment, the reactors 10, 110 have a width W of from about 500 mm to about 1,500 mm, a height H of from about 500 mm to about 2,000 mm, and a depth D of from about 500 mm to about 1,500 mm. In one illustrated embodiment, the width is about 1,100 mm, the height is about 1,500 mm, and the depth is about 1080 mm.

The reactors 10, 110 may be made of different materials. According to one embodiment, the reactor is made of metallic material such as stainless steel. More specifically, the top cover, the support base, and the at least one wall comprise stainless steel. It is contemplated that other materials may be used in forming the top cover, the support base, and the wall(s).

### Methods

The methods of the present invention are advantageous in reactions that require temperature control and/or controlled homogenous mixing. These methods avoid disadvantages that can occur from manually shaking the bag or package, while still producing desirable product and yield.

A method of using a reactor for an enzymatic reaction includes providing a reactor. The reactor includes a support base, at least one wall, a top cover and a moveable plate assembly. The top cover, the support base, and the at least one wall form an internal chamber therein that is hermetically closed. The moveable plate assembly including a moveable plate is located within the internal chamber. The moveable plate assembly is configured to move in the x-direction, y-direction, and the z-direction. The internal chamber of the reactor is accessible via at least one of the top cover, the support base, and the at least one wall. The bag is placed on the moveable plate assembly in a secured position. The bag includes at least one reaction component and an enzyme. The moveable plate is heated to a desired temperature. The temperature is controlled within the internal chamber. The moveable plate is moved in the x-direction, y-direction and the z-direction so as to mix the at least one reaction component and the enzyme.

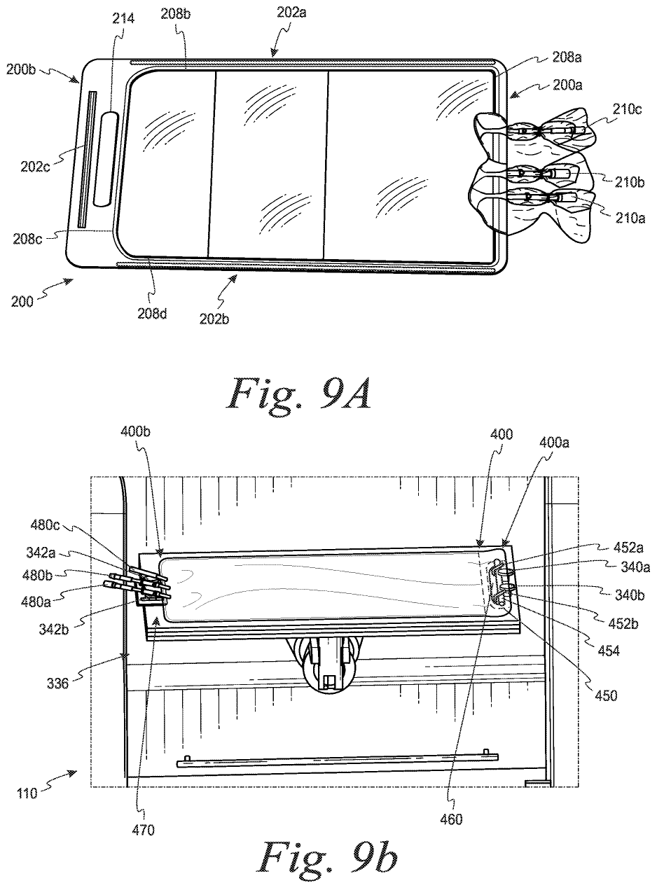

One example of a bag or package 200 that may be used in the reactors 10, 110 is shown in FIG. 9A. This bag or package 200 may be described as a bioprocess container (BPC). The bag or package 200 may vary in size, but typically is from about 1 to about 20 liters. It is contemplated that the bag or package may be smaller than 1 liter or may be larger than 20 liters, including up to 50 liters. The bag 200 is shown as being generally rectangular in size. It is contemplated that the bag or package may be of other shapes and sizes. The bag or package is desirably sized and shaped to remain on the moveable plate during the reaction process, including the mixing.

The bag or package 200 of FIG. 9A includes two elongated fixing sticks 202a, 202b and a shortened stick 202c. These sticks 202a-202c assist in securing the bag 200 to the moveable plate assembly. The bag of package 200 could be used, for example, with the moveable plate assembly 234 of FIG. 5A. It is contemplated that less sticks may be used for securing the bag. It is also contemplated that the length of the sticks may vary.

The bag or package 200 has a first end 200a and a second opposing end 202b. At the first end 200a, a plurality of injectors or syringes 210a-210c are shown. The injectors or syringes 210a-210c are luer lock injectors or syringes in one embodiment. It is contemplated that the injectors or syringes may be other types. The injectors or syringes 210a-210c enable additional components to be added to the package 200 during the process. Thus, the injectors or syringes 210a-210c are a conduit for these additional components to be directly entered into the interior of the bag or package 200. The interior of the bag or package 200 does not extend to the sticks 202a-202c, but are sealed along a perimeter identified in FIG. 9A by perimeter sections 208a-208d.

The shortened stick 202c and an aperture 214 are shown at the second end 200b. The combination of the shortened stick 202c and an aperture 214 with a bracket fixture (not shown) assist in securing the bag or package to a moveable plate. The elongated fixing sticks 202a, 202b extend generally between the first end 200a and the second opposing end 202b.

Referring to FIG. 9B, a bag or package 400 is shown in the reactor 110. The bag 400 has a first end 400a and a second end 400b. The bag or package 400 is secured on the moveable plate 336 using the pair of knobs 340a, 340b and 342a, 342b. The pair of knobs 340a, 340b includes an adjustable bracket 450 secured thereto. The adjustable bracket 450 includes a pair of upwardly and outwardly extending hooks 452a, 452b. The pair of upwardly and outwardly extending hooks 452a, 452b extends through an aperture 460 formed at the end 400a of the bag 400. The hooks 452a, 452b in combination with the pair of knobs 340a, 340b secure a fixing stick 454 located inside an opening of the bag 400. The opening is located adjacent to the aperture 460.

The pair of knobs 342a, 342b at the second end 400b includes an adjustable bracket 470 secured attached thereto. The second end 400b of the bag or package 400 is secured underneath of the adjustable bracket 470. At the second end 400b of the bag or package 400, a plurality of injectors 480a-c is shown. These contain components to be added to the bag or package 400.

The reactors of the present invention may carry out biochemical and/or chemical processes that require temperature control and/or controlled homogeneous mixing. It is contemplated that other gases may be used in the reactor to potentially improve enzymatic reactions. One non-limiting example of a gas that may be used is nitrogen.

In some embodiments of the invention, the reactor or bioreactor may assist in a portion of the biochemical and/or chemical processes such as before the purification steps. Non-limiting examples of biochemical processes that are carried out using the inventive reactor include enzymatic processes that involve at least one enzyme, in which the enzyme can be any enzyme known in the art. Some non-limiting enzymatic processes include restriction digestion with restriction endonucleases; nucleic acid (DNA and/or RNA polymerization); nucleic acid ligation (e.g., DNA ligation); reactions with telomerase or protelomerase; reactions with endonuclease and/or exonuclease; reactions with DNA polymerase or any variant thereof (e.g., with Klenow fragment); reactions with reverse transcriptase; reactions with recombinase; reaction with protease; reaction with nuclease; polymerase chain reaction; nucleic acid (e.g., DNA, complementary DNA, RNA) amplification; phosphorylation reaction; dephosphorylation reaction; nucleic acid synthesis reaction (e.g., oligonucleotide synthesis or primer synthesis); reaction with RNA polymerase; nick or damage repair reaction of nucleic acid; nucleic acid denaturation reaction; nucleic acid annealing reaction; nucleic acid extension reaction; or any combination thereof.

Non-limiting examples of at least one enzyme are restriction endonucleases, DNA polymerase, or any fragment thereof e.g., Klenow fragment, RNA polymerase, Phi 29 DNA polymerase, protelomerase (e.g., TelN, TelK), exonuclease, endonuclease, DNA recombinase, topoisomerase, Cre recombinase, reverse transcriptase, DNA ligase, meganucleases, zinc finger nucleases (ZFNs), transcription activator-like effector-based nucleases (TALEN), Cas9 nuclease and any combinations thereof. In several embodiments, the bag includes one or more enzymes known in the art, such as, for example, one or more enzymes selected from the group consisting of restriction endonucleases, DNA polymerase, or any fragment thereof e.g., Klenow fragment, RNA polymerase, Phi 29 DNA polymerase, protelomerase (e.g, TelN, TelK), exonuclease, endonuclease, DNA recombinase, topoisomerase, Cre recombinase, reverse transcriptase, DNA ligase, meganucleases, zinc finger nucleases (ZFNs), transcription activator-like effector-based nucleases (TALEN), Cas9 nuclease, and any combination thereof.

In some embodiments, the enzymatic reaction that are carried out by the bioreactor of the invention uses any nucleic acid known in the art as a substrate of the enzyme. In some embodiments, the enzymatic reaction that is performed in the bioreactor uses any protein or polypeptide known in the art as a substrate of the enzyme. In one aspect of the embodiment, the protein or polypeptide substrate is an antibody or a fragment thereof. In one embodiment, the nucleic acid modification (e.g., mutation, substitution, insertion, or deletion reaction) is performed using the bioreactor of the invention. In another embodiment, the bioreactor of the invention carries out methylation, demethylation, phosphorylation, dephosphorylation, or glycosylation reaction of nucleic acid and/or protein. In yet another embodiment, glycation of a protein is performed using the bioreactor of the invention. In some embodiments of the invention, the bioreactor is used for messenger RNA (mRNA) production.

In certain embodiments, the bioreactor of the invention is used to perform the modification of antibody. This may include, for example, antibody enzyme labeling, antibody nanoparticle labeling, antibody fragmentation, antibody biotinylation, antibody liposome conjugation, antibody immobilization or antibody oligonucleotide conjugation.

In some embodiments, the bioreactor of the invention is used for one or more steps of vaccine manufacturing process. Non-limiting examples of vaccine are Influenza vaccine, Covid-19 vaccine, Hepatitis A vaccine, Hepatitis B vaccine, HPV vaccine, Polio vaccine, Rabies vaccine, diptheria vaccine, Rotavirus vaccine, Rubella vaccine, shingles vaccine, smallpox vaccine, tetanus vaccine, Meningococcal vaccine, Varicella vaccine, and Pneumococcal vaccine.

In some embodiments, the bioreactor of the invention is used to perform gene editing reaction using any one of meganucleases, zinc finger nucleases (ZFNs), transcription activator-like effector-based nucleases (TALEN), and the clustered regularly interspaced short palindromic repeats (CRISPR/Cas9) system. The gene editing reaction repairs a double strand break in DNA by non-homologous end joining (NHEJ) or by homology directed repair (HDR), and thus either creates gene disruption by insertion or deletion, or create precise DNA editing. In certain embodiments, the bioreactor of the invention is used to perform conditional targeting of genes using site specific recombinases (e.g., Cre-loxP or Flp-FRT).

In some embodiments, the bioreactor of the invention is used for chemical reaction (e.g., in organic chemical synthesis reaction to synthesize small molecules) conjugation of probes using click chemistry, attaching larger macromolecule such as PEGylation, producing azo-compound, and the like. In the aspects of using the bioreactor of the invention for performing chemical reaction that require temperature control and/or homogeneous mixing may or may not need an enzyme as a reaction component.

In some embodiments, the enzymatic reactions that are performed using the bioreactor of the invention are performed at or above room temperature. For example, the temperature may be at least about 25° C., at least about 30° C., at least about 32° C., at least about 37° C., at least about 40° C., at least about 50° C., at least about 60° C., at least about 70° C., at least about 90° C., at least about 95° C., at least about 100° C. or even higher. In some embodiments, the enzymatic reactions that are performed using the bioreactor of the invention is performed at below room temperature. For example, the temperature may be below about 25° C., below about 20° C., below about 15° C., at below about 10° C., below about 5° C., below about 4° C., or below about 0° C.

Thus, the at least one reaction component in the bag or package is a nucleic acid in some embodiments. Some non-limiting examples of nucleic acids that can be used include DNA, RNA, oligonucleotide, inhibitory nucleic acids (e.g., miRNA, siRNA, RNAi, shRNA, dsRNA or variants thereof). Some non-limiting examples of DNA include cell free DNA, purified or isolated DNA, nicked or damaged DNA, modified DNA (e.g., DNA comprising substitution, insertion, deletion or any other modification such as, for example, methylation, demethylation, phosphorylation, dephosphorylation, addition of sugar moiety, conjugation of larger macromolecule such as polyethylene glycol, comprising chemical modification, or conjugation of labels or dyes). Some non-limiting examples of RNA include messenger RNA (mRNA), total RNA, transfer RNA (tRNA), guide RNA (gRNA), RNA comprising substitution, insertion, deletion or any other modification (e.g., methylation, demethylation, phosphorylation, dephosphorylation, addition of sugar moiety, conjugation of larger macromolecule such as polyethylene glycol, comprising chemical modification, or conjugation of labels or dyes). Some examples of modified DNA and/or RNA include DNA and/or RNA comprising modified bases (e.g., 2′-O-methoxy-ethyl Bases, 2′ O Methyl RNA bases, fluoro bases, 2 Aminopurine, 5 bromo dU, deoxyuridine or the like. In some embodiments, the at least one reaction component in the bag or package is a protein, polypeptide or the like. Some non-limiting examples of polypeptide or protein include isolated or purified protein, semi purified protein, proteins or polypeptides that are phosphorylated, dephosphorylated, methylated, demethylated, glycated or glycosylated. In some embodiments of the invention, the at least one component in the bag or package is oligonucleotide, primer, or probe. In some embodiments of the invention, the at least one component in the bag or package is deoxynucleoside triphosphates (dNTPs).

In the aspects of the embodiments described herein, the bioreactor of the invention produces consistent results with minimal variability. Thus, it may be suitably used for various biochemical and/or chemical processes where the comparability protocols may be successfully performed minimizing the variability occurring from the temperature variance and/or inconsistent mixing of reaction components.

In some embodiments, the bioreactor of the invention (also referred to as an inventive reactor) minimizes the variability between different batches of a reaction by at least about 25%, by at least about 20%, by at least about 15%, by at least about 10%, by at least about 5%, by at least about 3%, by at least about 2.5%, by at least about 2%, or by at least about 1% than when the different batches of the reaction are performed manually. In some embodiments, the bioreactor of the invention minimizes the variability between different batches of a reaction by about 2% to about 10%, about 5% to about 15%, about 10% to about 20%, about 15% to about 25%, about 20% to about 30%, about 25% to about 40% or even more than when the different batches of the reaction are performed manually.

The less variability between batches of a reaction using the bioreactor of the invention has immense value including for manufacturing clinical products. The regulatory authorities across the globe desire less variability between different production batches and, thus, the bioreactor of the invention can help adhere to regulatory compliance of a product that is produced in different batches using the bioreactor of the invention. Non-limiting examples of product may include, for example, close ended linear duplexed DNA (clDNA or celDNA), no end DNA (neDNA), or viral vectors such as AAV vectors manufactured using clDNA or neDNA as a template. Close ended linear duplexed DNA (clDNA or celDNA) is alternatively known as neDNA. Close ended linear duplexed DNA (clDNA or celDNA) is a linear double stranded DNA having closed ends, e.g., dbDNA. The less variability between batches is also desired in manufacturing products on an industrial scale.

In some embodiments, the at least one reaction component in the bag or package is a double-stranded (ds) or a single-stranded (ss) DNA. A double-stranded DNA may be an open circular double-stranded DNA, a closed circular double-stranded DNA, an open linear double-stranded DNA, or a closed linear double-stranded DNA. Preferably, the double-stranded DNA is a closed circular double-stranded DNA. In one embodiment of the invention, the at least one reaction component in the bag or package is plasmid DNA.

In some embodiments, the bioreactor is used to synthesize close ended linear duplexed nucleic acids. Non-limiting examples of close ended linear duplexed DNA (clDNA or celDNA), or neDNA that may be produced using the bioreactor of the present invention include doggy bone DNA (dbDNA) or dumbbell-shaped DNA. The close ended linear duplexed nucleic acids may be generated within cells or using in vitro cell free system. In some embodiments, the clDNA or neDNA that is produced using the bioreactor of invention also further comprises protelomerase binding site (e.g., TelRL). One such example of clDNA or neDNA is doggybone DNA or dbDNA. In certain embodiments of the invention, the enzyme in the bag is selected from the group consisting of Restriction Endonuclease, Phi 29 DNA polymerase, exonuclease, and any combination thereof. In some embodiments of the invention, the at least one reaction component in the bag or package is selected from the group consisting of plasmid DNA, oligonucleotide, deoxynucleoside triphosphate (dNTP), and any combination thereof. In some embodiments, the clDNA or neDNA that is produced using the bioreactor of the invention does not comprise a protelomerase binding site.

Non-limiting examples of methods that may be performed in one or more steps using the bioreactor of the invention include cell free in vitro synthesis of dumbbell-shaped DNA and doggy bone DNA as described in U.S. Pat. No. 6,451,563; Efficient production of superior dumbbell-shaped DNA minimal vectors for small hairpin RNA expression-Nucleic Acids Res. 2015 Oct. 15; 43(18): e120; High-Purity Preparation of a Large DNA Dumbbell-Antisense & nucleic acid drug development 11:149-153 (2001); U.S. Pat. Nos. 9,109,250; 9,499,847; 10,501,782; and WO 2018033730 A1; all of which are herein incorporated by reference in their entireties. The DNA from cell free in vitro synthesis is devoid of any prokaryotic DNA modifications (e.g., is substantially free of bacterial DNA). In some embodiments, the close ended linear duplexed DNA or neDNA synthesized using the bioreactor of the invention has less than 1% bacterial DNA.

One example of an in vitro process for producing a closed linear DNA (e.g., containing the ITRs described herein) or as used herein close ended linear duplexed DNA comprises (a) contacting a DNA template flanked on either side by a protelomerase target sequence with at least one DNA polymerase in the presence of one or more primers under conditions promoting amplification of said template; and (b) contacting amplified DNA produced in (a) with at least one protelomerase under conditions promoting formation of a closed linear expression cassette DNA. The closed linear DNA may be a closed DNA expression cassette DNA product that may comprise, consist or consist essentially of a eukaryotic promoter operably linked to a coding sequence of interest and, optionally, a eukaryotic transcription termination sequence. The closed linear expression cassette DNA product may additionally lack one or more bacterial or vector sequences, typically selected from the group consisting of: (i) bacterial origins of replication; (ii) bacterial selection markers (typically antibiotic resistance genes) and (iii) unmethylated CpG motifs.

As outlined above, any DNA template comprising at least one protelomerase target sequence may be amplified according to the bioreactor of the invention. Thus, although production of therapeutic DNA molecules (e.g., for DNA vaccines or other therapeutic proteins and nucleic acid) is preferred, the bioreactor of the invention may be used to produce any type of closed linear DNA. The DNA template may be a double stranded (ds) or a single stranded (ss) DNA. A double stranded DNA template may be an open circular double stranded DNA, a closed circular double stranded DNA, an open linear double stranded DNA, or a closed linear double stranded DNA. The template is desirably a closed circular double stranded DNA. Closed circular dsDNA templates are particularly preferred for use with RCA (rolling circle amplification) DNA polymerases. A circular dsDNA template may be in the form of a plasmid or other vector typically used to house a gene for bacterial propagation. Thus, using the reactor can amplify any commercially available plasmid or other vector, such as a commercially available DNA medicine, and then convert the amplified vector DNA into closed linear DNA. Phi29 DNA polymerase is used to amplify double-stranded DNA by rolling circle amplification, and a protelomerase to generate close ended linear duplexed DNA (e.g., dbDNA), which coupled with a streamlined purification process, results in a pure DNA product containing just the sequence of interest. Phi29 DNA polymerase has high fidelity (e.g., 1×10⁶-1×10⁷) and high processivity (e.g., approximately 70 kbp). These features make this polymerase particularly suitable for the large-scale production of GMP DNA. Protelomerases (also known as telomere resolvases) catalyze the formation of covalently closed hairpin ends on linear DNA and have been identified in some phages, bacterial plasmids and bacterial chromosomes. A pair of protelomerases recognizes inverted palindromic DNA recognition sequences and catalyzes strand breakage, strand exchange and DNA ligation to generate closed linear hairpin ends. The formation of these close ended linear duplexed structures makes the DNA resistant to exonuclease activity, allowing for simple purification and can improve stability and duration of expression. The bioreactor of the invention is used to perform rolling circle amplification and/or protelomerase reaction, or a combination thereof to generate close ended linear duplexed DNA or neDNA (e.g., dbDNA).

An open circular dsDNA may be used as a template where the DNA polymerase is a strand displacement polymerase that can initiate amplification from at a nicked DNA strand. In this embodiment, the template may be previously incubated with one or more enzymes that nick a DNA strand in the template at one or more sites. A closed linear dsDNA may also be used as a template. The closed linear dsDNA template (starting material) may be identical to the close ended linear duplexed DNA product. Where a closed linear DNA is used as a template, it may be incubated under denaturing conditions to form a single stranded circular DNA before or during conditions promoting amplification of the template DNA.

In one example, the reactor or bioreactor as described in this invention is used to perform the rolling circle amplification (RCA) process to generate amplified DNA followed by digestion of the amplified DNA with restriction enzymes, and further contacting the digested form of amplified DNA with at least one protelomerase, in an automated way with controlled temperature and controlled homogeneous mixing conditions and, thus, forming doggy bone DNA (dbDNA). In this example, the template used for RCA may be a nucleic acid such as open circular dsDNA or closed linear dsDNA. The RCA process comprises denaturation of the template DNA followed by contacting with oligonucleotides (serving as primers), dNTP (deoxynucleoside triphosphate), and another enzyme (Phi29 polymerase). In this embodiment, a buffer is typically included.

The duration, speed, angles and phase delay of the moveable plate as well as the incubation temperature of the reactor can vary depending on the reaction. In one non-limiting example of performing a reaction for forming clDNA or neDNA, the temperature varies from about 25 to 40 degrees Celsius. The incubation times can vary from minutes to hours to get the proper steps completed in a desirable fashion. For example, the RCA step may be incubated from about 25 to 35 hours and the RE step may be from about 10 to about 80 hours. The TelN steps may be incubated from about 15 minutes to about 35 minutes, while the exonucleases processing may be incubated from about 15 to about 25 hours.

The moveable plate in the inventive reactor typically oscillates at a speed of from about 10 to about 50 rpm with angles from about 10 to about 20 degrees with different phases (0, 90 and 180 degrees) in reactions for forming clDNA or neDNA.

Thus, in one method, the components include a buffer, a plurality of enzymes, nucleic acid template, deoxynucleotide triphosphates (dNTPs) and at least one primer.

The reactants for specific reactions are delivered to the bag used in the reactor either subsequently to one another, or, in different combinations. After each incubation of the reactions at desired temperature, e.g., after certain time intervals, the reactants of the next reactions are added to the bag.

In certain embodiments the bioreactor of the invention is used to perform one or more steps of alternate methods of generating covalently closed end linear DNA that lack bacterial sequences e.g., by formation of mini-circle DNA from plasmids as described in U.S. Pat. Nos. 8,828,726 and 7,897,380, the contents of each of which is incorporated by reference in their entirety. For example, one method of cell-free synthesis combines the use of two enzymes (Phi29 DNA polymerase and a protelomerase) and generates high fidelity, covalently close ended linear duplexed DNA constructs. The constructs contain no antibiotic resistance markers and therefore eliminate the packaging of these sequences.

In some embodiments, the bioreactor of the invention is used to perform one or more steps of generating covalently closed DNA as described in U.S. Publication No. 2020/0362403, which is incorporated hereby by reference in its entirety. The bioreactor is used for all of the following steps or, any one or any combination of the following steps that comprise pretreating the linear double-stranded DNA template (e.g., removing the protruding ends; restoring 5′Phosphate and 3′OH groups; adding the 3′ dA overhangs; ligating the hairpin adapter at 5′ and 3′ end followed by forming a single-stranded covalently closed DNA that undergoes rolling circle amplification; and multiple strand displacement by a combination of enzymes Primase-Polymerase and Phi29 Polymerase).

Phi29 DNA polymerase is used to amplify double-stranded DNA by rolling circle amplification, and a protelomerase to generate covalently close ended linear duplexed DNA, which coupled with a streamlined purification process, results in a pure DNA product containing just the sequence of interest. Phi29 DNA polymerase has high fidelity Phi29 DNA polymerase has high fidelity (e.g., 1×10⁶-1×10⁷) and high processivity (e.g., approximately 70 kbp). These features make this polymerase particularly suitable for the large-scale production of GMP DNA. Protelomerases (also known as telomere resolvases) catalyze the formation of covalently closed hairpin ends on linear DNA and have been identified in some phages, bacterial plasmids and bacterial chromosomes. A pair of protelomerases recognizes inverted palindromic DNA recognition sequences and catalyzes strand breakage, strand exchange and DNA ligation to generate close ended linear hairpin ends. The formation of these close ended structures makes the DNA resistant to exonuclease activity, allowing for simple purification and can improve stability and duration of expression.

In one embodiment, the close ended linear duplexed DNA is produced in eukaryotic cells for example insect cells as described in PCT publications WO 2019032102 and WO 2019169233. In one embodiment, the DNA is not produced in eukaryotic cells and DNA lacks eukaryotic sequences. In one embodiment, the close ended linear duplexed DNA vectors are produced as described in PCT publication WO 2019143885.

In certain embodiments, an in vivo cell system is used to produce close ended linear duplexed nucleic acids, where one or more steps of the production method is carried out using the bioreactor. The method comprises using a cell that expresses a protelomerase, such as TelN, or other protelomerase, in which the protelomerase gene is under the control of a regulatable promoter. For example, an inducible promoter such as a small molecule regulated promoter or a temperature sensitive promoter (e.g., a heat shock promoter). After sufficient production of the nucleic acid of interest, or combination thereof, one can allow the protelomerase to be expressed, which will excise the nucleic acid of interest from the template.

In certain embodiments, the in vivo cell system is used to produce a non-viral DNA vector construct for delivery of a predetermined nucleic acid sequence into a target cell for sustained expression. The non-viral DNA vector comprises, two DD-ITRs each comprising: an inverted terminal repeat having an A, A′, B, B′, C, C′ and D region; a D′ region; and wherein the D and D′ region are complementary palindromic sequences of about 5-20 nt in length, are positioned adjacent the A and A′ region; the predetermined nucleic acid sequence (e.g. a heterologous gene for expression); wherein the two DD-ITRs flank the nucleic acid in the context of covalently closed non-viral DNA and wherein the closed linear vector comprises a ½ protelomerase binding site on each end.

The system comprises recombinant host cells. Suitable host cells for use in the production system include microbial cells, for example, bacterial cells such as E. coli cells, and yeast cells such as S. cerevisiae. Mammalian host cells may also be used including Chinese hamster ovary (CHO) cell for example of K1 lineage (ATCC CCL 61) including the Pro5 variant (ATCC CRL 1281); the fibroblast-like cells derived from SV40-transformed African Green monkey kidney of the CV-1 lineage (ATCC CCL 70), of the COS-1 lineage (ATCC CRL 1650) and of the COS-7 lineage (ATCC CRL 1651, murine L-cells, murine 3T3 cells (ATCC CRL 1658), murine C127 cells, human embryonic kidney cells of the 293 lineage (ATCC CRL 1573), human carcinoma cells including those of the HeLa lineage (ATCC CCL 2), and neuroblastoma cells of the lines IMR-32 (ATCC CCL 127), SK-N-MC (ATCC HTB 10) and SK-N-SH (ATCC HTB 11). The host cell is designed to encode at least one recombinase. The host cell may also be designed to encode two or multiple recombinases. The term “recombinase” refers to an enzyme that catalyzes DNA exchange at a specific target site, for example, a palindromic sequence, by excision/insertion, inversion, translocation and exchange. Examples of suitable recombinases for use in the present system include, but are not limited to, TelN, Tel, Tel (gp26 K02 phage) Cre, Flp, phiC31, Int and other lambdoid phage integrases, e.g., phi 80, HK022 and HP1 recombinases.

The volume of the bag or package can vary, but is generally from about 50 ml to about 10 L or about 20 L. It is contemplated that the volume may be smaller or larger. The volume of the bag or package is typically from about 500 ml to about 10 L.

The inventive reactor used for incubation and automated agitation during different enzymatic reaction steps may use different scaling. Thus, the volume of the performed reactions may vary in the inventive reactor. Some typical volume sizes include 20 ml and 500 ml. It is contemplated that larger volume sizes, including 1500 ml and 5000 ml, may be performed in the inventive reactor. The volume of the reactions performed in the inventive reactor is generally from about 10 ml to about 10 L in one embodiment. The volume of the reactions performed in the inventive reactor is from about 10 ml to about 5000 ml in another embodiment. The volume of the reactions performed in the inventive reactor is from about 10 ml to about 2000 ml in a further embodiment. The volume of the reactions performed in the inventive reactor is from about 10 ml to about 1000 ml in a further embodiment. The volume of the reactions performed in the inventive reactor is from about 20 ml to about 500 ml in yet another embodiment.

The temperature of the internal chamber may be heated and controlled by different methods. In one embodiment, the temperature of the internal chamber is heated by a resistive heating element layer mounted on a bottom of the support base. The temperature of the internal chamber is controlled in one embodiment by a plurality of probes located thereon such as, for example, on one of the sidewalls. These plurality of probes may be on opposing sidewalls. The probes used may be the same as described above with respect to the moveable plate. It is contemplated that the temperature of the internal chamber may be heated and controlled by other methods.

If ambient temperature is desired within the internal chamber in another embodiment, a plurality of ducts may carry desired air into the reactor. In one embodiment, the plurality of ducts may each be equipped with a fan. In other embodiment, the temperature of the internal chamber of the reactor may be cooled to a desired temperature. In such an embodiment, a cooling system would need to be added and integrated within the reactor. In one embodiment, the cooling system may be a water-based cooling system. In another embodiment, the cooling system may be an air-based cooling system.

### EXAMPLES

Several examples were performed implementing and using the inventive reactor described above with respect to FIGS. 1-4 and 6 that used a locking feature and HEPA H14 filters. The inventive reactor was used for incubation and automated agitation during different enzymatic reaction steps using different scaling (20 ml and 500 ml). These inventive reactor examples with automatic agitation were compared to incubator examples that were manually agitated. The control or comparative incubator was either BINDER incubator KT115 or BD115.

The examples using the inventive reactor or the comparative incubator were taken from different enzymatic reaction steps in closed linear DNA (clDNA) manufacture: (i) Phi29 polymerase for Rolling Circle Amplification (RCA), (ii) Restriction Enzymes (RE) to break up the DNA concatemer, (iii) TelN protelomerase for covalent closure of both the DNA ends, and (iv) exonucleases for removing DNA impurities.

Each of these enzymatic reaction steps had different incubation times, temperatures, and agitation frequencies. In the control or comparative incubator examples, agitation was performed manually by individuals with ×10 inversions and ×10 orbital shaking of the reaction bag to ensure the homogeneity of the solution and components availability for the different enzymes.

To reproduce the manual movements in the comparative incubator, an independent program for each enzymatic reaction step was designed for the inventive reactor for automated agitation. The plate movements of the inventive reactor were designed to achieve homogeneity of the reaction in a bag, considering the solution viscosity along the process. The solution's viscosity changed along the different reaction steps. The solution's viscosity reaches its maximum at the end of the RCA step and decreases after Restriction Enzyme (RE) addition. The program conditions, such as temperature and agitation frequency, were generally based on close ended linear duplexed DNA (clDNA) production.

### Example 1—Experiment of clDNA Yield in Reactor Automated Agitation Process During RCA Step (20 ml Scale)

Each enzymatic reaction was assessed separately. The experimental method was common in these experiments: 20 ml reactions were performed in 50 ml single-use bioprocess containers (filling ratio: 0.400) in all reaction steps including the RCA step. Each experimental condition was performed in triplicate for statistical analyses, resulting in 6 total reactions in Example 1.

To demonstrate that the inventive reactor can substitute comparative incubators with manual agitation, the RCA step was initially tested. During this assay, the yields were observed and analyzed between (1) incubating and automatedly agitating the reaction bag in the inventive reactor, and (2) agitating manually in the comparative incubator during the RCA step. The remaining enzymatic steps were not assessed in Example 1, but rather were completed using the comparative incubator with agitations performed manually. In other words, after the RCA step, the RE step, TelN protelomerase step, and exonuclease step were performed by manual agitation in the comparative incubator.

After exonucleases processing, the clDNA yield was quantified by Quantifluor® dsDNA analysis and a two-tailed t-student test was calculated (95% confidence interval (CI)). The results showed that automated agitation during the RCA step did not have a statistically significant impact on the clDNA yield when compared to manual agitation using the comparative incubator. See Table 1 below.

Specifically, the average clDNA yield of triplicate reactions incubated and automatedly agitated in the inventive reactor was 0.109±0.009 mg/ml, while the average clDNA yield of triplicate reactions agitated manually in the comparative incubator was 0.104±0.010 mg/ml as shown in Example 1.

### Example 2—Experiment of clDNA Yield in Reactor Automated Agitation Process During the RCA and Restriction Enzyme Steps (20 ml Scale)

After demonstrating that automated agitation during the RCA did not have a significant effect on the clDNA yield, the inventive reactor was tested during the Restriction Enzyme (RE) step. In this assay, the yield was observed and analyzed between (1) the incubation and automated agitation in the inventive reactor during both the RCA and Restriction Enzyme (RE) steps and (2) agitating manually in the comparative incubator in these steps. The remaining enzymatic steps were not assessed in Example 2, but rather were completed using the comparative incubator with agitations performed manually. In other words, after the RCA and RE steps, the TelN protelomerase and exonuclease steps were performed by manual agitation in the comparative incubator.

The testing conditions in Example 2 included: (a) automatic agitation during the RCA and RE steps; (b) automatic agitation during the RCA step, but manual agitation during the RE step; (c) manual agitation during the RCA step, but automatic agitation during the RE step; and (d) manual agitation during the RCA and RE steps. Each condition was performed in triplicate, resulting in 12 total reactions in Example 2.

After Restriction Enzyme (RE) processing, a 0.8% agarose gel was used to check if the reactions were correctly digested. All reactions showed the expected band pattern (9005 bp, 969 bp and 860 bp bands).

The results after exonuclease processing are shown in Table 2 below. The final clDNA yield was quantified by Quantifluor® dsDNA analysis and a two-tailed t-student test was calculated (95% CI). The results showed that the inventive reactor with automated agitation can substitute manual agitation in a comparative incubator during both the RCA and Restriction Enzyme (RE) steps without having a statistically significant impact on clDNA yield.

The average clDNA yield of triplicate reactions incubated and agitated in the inventive reactor was 0.58±0.11 mg/ml, while the average clDNA yield of triplicate reactions agitated manually was 0.56±0.08 mg/ml.

### Example 3—Experiment of clDNA Yield in Reactor Automated Agitation Process (20 ml Scale) During all Enzymatic Steps

After demonstrating that automated agitation during both RCA and Restriction Enzyme (RE) steps did not have a significant effect on the clDNA yield, the inventive reactor with automated agitation was tested during the TelN and Exonuclease steps. During this assay, the yields and purities were compared between (1) incubating and automatedly agitating the reaction bag in the inventive reactor, and (2) agitating manually in the comparative incubator during the TelN and Exonuclease steps. The previous enzymatic reaction steps were not assessed in this assay, but incubated and automatedly agitated in the inventive reactor.

The testing conditions were: (a) inventive reactor incubation and automatic agitation in TelN and exonuclease steps; (b) comparative incubator using manual agitation in TelN and exonuclease steps. Each condition was performed in triplicate for each condition and construct, resulting in 12 total reactions in Example 3.

After TelN processing, two parallel 0.8% agarose gels were used to check if the two constructs were correctly processed. All reactions showed the expected band pattern after TelN processing. For the first construct, a DNA band was observed at the expected size (8884 bp) and some backbone fragments were observed (969 bp and 860 bp) as expected. For the second construct, a DNA band was observed at the expected size (4572 bp) and some backbone fragments were observed (813 bp, 799 bp, 203 bp and 201 bp) as expected.

The clDNA yields were quantified by Quantifluor® dsDNA analysis and clDNA purity was analyzed by calculating the raw volume % of the clDNA by AGE densitometry. A two-tailed t-student was calculated (95% CI) in both analyses. The results showed that the inventive reactor with automated agitation can substitute for the comparative incubator with manual agitation during all the clDNA manufacturing enzymatic reactions steps without having a statistically significant impact on the clDNA yield and/or purity at a 20 ml scale. See Tables 3 and 4 below.

The average clDNA yield of triplicate reactions incubated and automatedly agitated in the inventive reactor during the entire process was 0.53±0.05 mg/ml and 0.58±0.04 mg/ml as shown in Table 3. The average clDNA yield of triplicate reactions agitated manually in the comparative incubator during TelN and Exonuclease steps was 0.46±0.06 mg/ml and 0.62±0.07 mg/ml as shown in Table 3.

In Table 4, the average clDNA purity of triplicate reactions incubated and automatedly agitated in the inventive reactor during the entire process was 83.38±4.23% and 88.44±1.23%. The average clDNA purity of triplicate reactions agitated manually during the TelN and Exonuclease steps was 90.66±2.88% and 90.70±2.80%.

### Example 4—Experiment of clDNA Yield and Purity in Reactor Automated Agitation Process During all Enzymatic Steps (500 ml Scale)

After demonstrating that yield and purity of clDNA manufactured with the inventive reactor at 20 ml scale reactions is maintained (Examples 1-3), an evaluation was performed by scaling-up to 500 ml scale during the reaction steps.

Two parallel 500 ml scale clDNA manufacturing batches in 1 L single-use bioprocess containers (filling ratio: 0.445) were performed. The first batch was performed using the inventive reactor with automated agitation as described above. The second batch was performed using the comparative incubator that was manually agitated.

At the end of all enzymatic steps, an agarose gel was used to check if the steps were correctly processed. The automated and manual reactions showed the expected band pattern: Restriction Enzyme (RE) digested reactions completely, TelN closed the clDNA covalently, and exonucleases processed all the backbone fragments. After exonuclease processing, the final products from both reactions were analyzed to check identity and compare their final clDNA yields and purities.

The identity of the final product clDNA was confirmed by agarose gel analysis and DNA sequencing. Results from the agarose gel analysis confirmed that both reactions (inventive reactor with automated agitation and comparative incubator with manual agitation) resulted in a final product with the expected band size (4572 bp±10%). Sequencing analysis confirmed that both products have a 100% coincidence with the reference sequence. Overall, the results from these analyses showed that the inventive reactor with automated agitation can substitute manual agitation during all the clDNA manufacturing steps at a 500 ml scale without having an impact on the clDNA identity.

The clDNA yield was quantified by Quantifluor® dsDNA analysis. The two-tailed t-student test (95% CI) was used and comparison between both reactions showed that the inventive reactor agitation with automated agitation can substitute manual agitation during all the clDNA manufacturing enzymatic process at a 500 ml scale without having a significant impact on the clDNA yield. See Table 5 below.

The clDNA yield of reaction incubated and agitated in the inventive reactor during the total process resulted in 0.48 mg/ml. The clDNA yield of reaction incubated in the control incubator and agitated manually during all enzymatic steps resulted in 0.54 mg/ml.

The clDNA purity was analyzed by calculating the raw volume % of the clDNA by AGE densitometry and a two-tailed t-student test (95% CI) was calculated. For an in-depth clDNA purity analysis, qPCR testing was performed. In summary, comparison of the AGE densitometry and qPCR results between both reactions (inventive reactor with automated agitation and control incubator with manual agitation) showed that the inventive reactor with automated agitation can substitute manual agitation during all the clDNA manufacturing enzymatic steps at a 500 ml scale without having a significant impact on the clDNA purity. See Table 6 below.

The clDNA purity of reaction incubated and agitated in the inventive reactor during the total process was 84.41%. The clDNA purity of reaction incubated in the control incubator and agitated manually during all enzymatic steps was 86.26%.

In conclusion, the inventive reactor incubation with automated agitation can substitute manual agitation during all the clDNA or neDNA manufacturing enzymatic reaction steps at a 20 ml scale, without having a statistically significant impact on the clDNA or neDNA identity, yield or purity. The inventive reactor incubation with automated agitation can substitute manual agitation during all the clDNA or neDNA manufacturing enzymatic reaction steps at a 500 ml scale without having a statistically significant impact on the clDNA or neDNA identity, yield or purity.

### Example 5—Inventive Bioreactor is Used to Manufacture the Starting Materials for Recombinant AAV Production

The inventive bioreactor is used to manufacture the starting materials for recombinant AAV production, such as: (i) Ad helper clDNA or neDNA (e.g., Ad helper dbDNA), (ii) AAV Rep-Cap helper clDNA or neDNA (e.g., AAV helper Rep-Cap dbDNA), and (iii) AAV clDNA or neDNA encompassing transgene flanked by AAV ITRs (e.g., AAV dbDNA genome encoding the transgene flanked by AAV ITRs). The inventive bioreactor produces the (i), (ii), and (iii) final products with the desired specifications, all within the acceptance criteria as stated in Table 7 below. The starting materials (i), (ii), (iii) were produced from different reaction volumes, e.g., 20 ml, 445 ml, 500 ml, 1335 ml, or 4450 ml for each of the volumes tested the final product of (i), (ii) and (iii) are within the acceptance criteria as stated in Table 7 below.

TABLE 7

| | Acceptance | | | | | | | | | | | |
|---|---|---|---|---|---|---|---|---|---|---|---|---|
| Parameter | Criteria | | Batch 1 | | Batch 2 | | Batch 3 | | Batch 4 | | Batch 5 | |
| Visual | Transparent, | | Transparent, | | Transparent, | | Transparent, | | Transparent, | | Transparent, | |
| Appearance | colourless | | colourless | | colourless | | colourless | | colourless | | colourless | |
| | liquid without | | liquid without | | liquid without | | liquid without | | liquid without | | liquid without | |
| | extraneous | | extraneous | | extraneous | | extraneous | | extraneous | | extraneous | |
| | particulates | | particulates | | particulates | | particulates | | particulates | | particulates | |
| Concentration | 0.80-1.50 | mg/mL | 1.26 | mg/mL | 1.23 | mg/mL | 1.28 | mg/mL | 1.32 | mg/mL | 1.21 | mg/mL |
| Identity A: | 100% match | | 100% match | | 100% match | | 100% match | | 100% match | | 100% match | |
| DNA | with the | | with the | | with the | | with the | | with the | | with the | |
| sequence | reference | | reference | | reference | | reference | | reference | | reference | |
| | sequence | | sequence | | sequence | | sequence | | sequence | | sequence | |
| Identity B: | Band at | | 4729 pb | | 4642 bp | | 4513 pb | | 9294 pb | | 4459 bp | |
| Size | expected | | Expected size: | | Expected size: | | Expected size: | | Expected size: | | Expected size: | |
| confirmation | size based | | 4572 bp | | 4569 bp | | 4572 bp | | 9367 pb | | 4569 bp | |
| | on reference | | | | | | | | | | | |
| | sequence | | | | | | | | | | | |
| | (±10%) | | | | | | | | | | | |
| Purity | ≥95.0% | | 100.0% | | 100.0% | | 99.8% | | 97.3% | | 99.4% | |
| Related | ≤5.0% | | 0.0% | | 0.0% | | 0.2% | | 2.7% | | 0.6% | |
| substances | | | | | | | | | | | | |
| Impurity A | ≤0.1% w/w | | <0.1% w/w | | N/A | | <0.1% w/w | | <0.1% w/w | | <0.1% w/w | |
| (Residual | TelN | | TelN | | | | TelN | | TelN | | TelN | |
| TelN) | | | (0.0008%) | | | | (0.0066%) | | (0.0074%) | | (0.0085%) | |
| Impurity B | ≤2% | w/w | 0.47% | w/w | N/A | | <0.49% w/w | | <1% w/w | | <1% w/w | |
| (Residual | | | | | | | | | (<0.47%) | | (<0.52%) | |
| Protein | | | | | | | | | | | | |
| Content) | | | | | | | | | | | | |
| Impurity C | 1.75-2.00 | | 1.91 | | 1.90 | | 1.91 | | 1.92 | | 1.90 | |
| (Relative | | | | | | | | | | | | |
| Protein | | | | | | | | | | | | |
| Content) | | | | | | | | | | | | |
| Endotoxin | ≤5.00 | IU/mg | <1.19 | IU/mg | <1.22 | IU/mg | <1.18 | IU/mg | <1.14 | IU/mg | <0.83 | IU/mg |

| Bioburden | TAMC < | TAMC < | | TAMC < | | TAMC < | | TAMC < | | TAMC < | |
|---|---|---|---|---|---|---|---|---|---|---|---|
| | 1 CFU/mL | 1 CFU/mL | | 1 CFU/mL | | 1 CFU/mL | | 1 CFU/mL | | 1 CFU/mL | |
| | TYMC < | TYMC < | | TYMC < | | TYMC < | | TYMC < | | TYMC < | |
| | 1 CFU/mL | 1 CFU/mL | | 1 CFU/mL | | 1 CFU/mL | | 1 CFU/mL | | 1 CFU/mL | |
| pH | 7.00-8.50 at | 7.88 (23° C.) | | 7.95 (23° C.) | | 8.01(24° C.) | | 7.99 (23° C.) | | 8.06 (24° C.) | |
| | 20-25° C. | | | | | | | | | | |
| Osmolality | Report result | 21 | mOsm/kg | 26 | mOsm/Kg | 19 | mOsm/Kg | 23 | mOsm/kg | 19 | mOsm/Kg |

### Example 6—Experiment on Yields and Purity of clDNA Using Inventive Reactor

A 445 ml close ended linear duplexed DNA (clDNA) run was performed using the inventive reactor described above with respect to FIGS. 1-4 and 6 that used a locking feature and HEPA H14 filters. This run was compared and confirmed with the final specifications for clDNA or neDNA. The specific clDNA or neDNA that was used in Example 6 was dbDNA.

Example 6 was taken from different enzymatic reaction steps in closed linear DNA (clDNA) manufacture: (i) Phi29 polymerase for Rolling Circle Amplification (RCA), (ii) Restriction Enzymes (RE) to break up the DNA concatemer, (iii) TelN protelomerase for covalent closure of both the DNA ends, and (iv) exonucleases for removing DNA impurities.

The RCA in this 445 ml clDNA run proceeded without any significant events related to the inventive reactor. A reagent feed was added to the reaction after 7 hours of amplification. The first 30° C. incubation of the bioprocess container (BPC) containing 1λTLG buffer (30 mM Tris-HCl pH 7.8; 30 mM KCl; 8 mM MgCl₂; 5 mM (NH₄)SO₄) for the RCA step was initiated in a binder incubator because the inventive reactor was not available.

After the amplification step, 300 U/mL of Xbal was added to the BPC. It is noted that the lot of Xbal contained lower amounts of units than the expected 1×10⁶ U/ml, which caused a high level of undigested fragments. This was later confirmed by the manufacturer. To address the lower amounts of units, the lot was reprocessed by adding an additional 300 U/ml of Xbal (RE). This additional amount of Xbal was incubated for at least 16 hours.

The expected agarose gel (AGE) post Xbal digestion was expected to be 4824 bp, 813 bp, 799 bp, and 203 bp. Due to the small difference in base pairs (bps), the 813 and 799 bp bands couldn't be clearly separated under the running conditions used. After the reprocessing step where the additional Xbal was added, the RCA reaction was well digested. The process continued with the TelN processing step after confirming the correct RE digestion of the RCA.

TelN protelomerase was then added to the BPC and incubated at 30° C. for 20 min. This enzyme was responsible for recognizing and binding to the TelN sites, and cleaving and covalently closing both ends. Thus, in this step, clDNA molecules were built. The expected AGE Pattern post TelN digestion was 4572 bp (clDNA), 813 bp, 799 bp, 203 bp, 201 bp and 51 bp.

The TelN protelomerase step proceeded well, as a light downward shift was observed in the agarose gel, which indicated good formation of the clDNA. Once the TelN processing step was verified as being correctly performed, the first downstream processing (DSP) step was carried out. This is also referred to as DSP1. A clarification step was performed using a 0.45 μm in-line filter, and then a salt adjustment to detach the TelN protelomerase from the clDNA molecules and prepare the sample for the purification step with the chromatography. For this next step, a multimodal chromatographic step was used. The flow-through fraction containing the clDNA and other species was then concentrated using a 30 kDa hollow fiber tangential flow filtration (TFF). The sample was then diafiltrated to new 1λTLG buffer, and topped up to 445 mL of 1λTLG, prior to adding the exonucleases. Exonucleases were added to the BPC to degradate ssDNA intermediate species and open dsDNA fragments. It is noted that since clDNA is a linear dsDNA molecule covalently closed at both ends, it cannot be digested by the exonucleases. Together with the exonucleases, 50 U/mL extra of Xbal (RE) was added. Additionally, a small fraction of the reaction where only exonucleases are added (with no extra clDNA processing RE), which is routine in most of clDNA runs and has no major impact in product or process.

All of the downstream processing (DSP1) reaction samples were shown per manufacturing instructions. After the DSP1 was verified as being properly run, a second downstream processing (DSP2) step was performed with the chromatography and TFF steps as discussed above in DSP1. The clDNA was finally diafiltrated to the final formulation buffer of clDNA; 10 mM Tris. The concentration of the clDNA bulk was adjusted to 1.27 mg/mL.

The results of DSP2 were all within expected parameters. It is noted that a tiny portion of clDNA was observed in both permeates of the DSP2 TFF as is sometimes observed among manufacturing batches. clDNA was also present in, but was not in sufficient quantity to be pooled with the formulated clDNA formed after the second tangential flow filtration (TFF2). Nevertheless, part of this fraction was later used to dilute and adjust the concentration of the clDNA within the established concentration as per manufacturing instructions. Some material was lost in TFF2/P1 and in TFF2/P2, which is expected and not usual for the 445 mL scale.

The final filtration of the clDNA product was performed under laminar flow conditions. For the filtration, a 0.22 μm in-line filter was used. A total yield of 135.97 mg of clDNA was obtained after the final filtration step. Light bands in the lanes that were not loaded showed a light band of the correct size that most likely come from the adjacent wells due to a technical issue when loading the gels. No problems were encountered during the final sterilizing 0.22 μm filtration of the clDNA. A loss of only 7.6% in mg was observed, which is within the range of filtration losses for this scale. In the clDNA final product agarose gel, no other bands appeared, and the purity of the product was 100%.

Regarding the yield obtained in Example 6, the 135.97 mg of final product obtained was close to the mean value (140.59 mg) obtained in 63 batches of the 445 mL scale manufactured previously not using the inventive reactor. This yield in Example 6 is within a 95% confidence interval (CI) [130.88,150.31] and well within the values estimated for the mean±/−2 standard deviations. Therefore, all analytics of the final product of clDNA showed that Example 6 complied with current specifications.

### Example 8: The Automated Reactions in the Inventive Reactor Provide Homogeneous Mixing Over Long Reaction Hours

One particular example of clDNA or neDNA is dbDNA that was manufactured using the inventive bioreactor. The different steps of producing the dbDNA comprise different enzymatic reactions and shown in Table 8 below, all of them cumulatively take a long time requiring homogeneous mixing under controlled temperature throughout the reaction steps for a successful production of the final product. The inventive reactor is programmed to implement the automation of the incubation and agitation of the different enzymatic reactions, thus ensuring a homogeneous mixing over long period of time, and leading to the successful production of the final product (e.g., dbDNA) with a minimum variability between production batches. On the contrary, manual mixing would be extremely laborious to achieve especially for larger volumes (e.g., for 4450 ml or higher), over lengthy enzymatic reactions, and would be prone to variability arising from inconsistent mixing over long period of time.

TABLE 8

| Current manual process | | | | Inventive | Programs | |
|---|---|---|---|---|---|---|
| Enzymatic | | Incubation | | Program | Incubation | |
| reaction | Temperature | time | Agitation steps | name | time | |
| Standard Rolling | 30° C. | 7.5 h ± | Initial mix (Time = 0 h) | RCA_FASE1 | 7-8 | hours |
| Circle | (Incubator | 0.5 h | Every 2 hours during the first six | | | |
| Amplification | EQ-034) | | hours (Times: 2 h ± 10 min; 4 h ± | | | |
| (stdRCA) | | | 10 min; 6 h ± 10 min) | | | |
| | | 21.5 h ± | Feed mix (Time: 7.5 h ± 0.5 h) | RCA_FASE2 | 21 | hours |
| | | 0.5 h | Two additional mixes at 24 and | | | |
| | | | 26 hours (Times: 24 h ± 10 min; | | | |
| | | | 26 h ± 10 min). | | | |
| Restriction | 37° C. | 16-67 | Initial mix (Time = 0 min) | RESTRICTION- | 16-67 | hours |
| Enzyme (RE) | (Incubator | hours | Every 15 minutes during the first | ENZYME | | |
| Digestion | EQ-036) | | hour (Times: 15 min ± 5 min; 30 | | | |
| | | | min ± 5 min; 45 min ± 5 min; 1 h ± | | | |
| | | | 5 min) | | | |
| | | | Every 30 minutes during the | | | |
| | | | second hour (Times: 1.5 h ± 5 | | | |
| | | | min; 2 h ± 5 min). | | | |
| | | | Every 2 hours from the 2^{nd} to the | | | |
| | | | 16^{th} hour (Times: 4 h, 6 h, 8 h, | | | |
| | | | 10 h, 12 h, 14 h and 16 h). | | | |
| Covalent closure | 30° C. | 20-25 | Initial mix (Time = 0 min) | TelN | 20-25 | minutes |
| of neDNA ™ by | (Incubator | minutes | | | | |
| TelN | EQ-034) | | | | | |
| Exonucleases | 37° C. | 16-20 | Initial mix. | EXOS | 16-30 | hours |
| processing | (Incubator | hours | Second mix (Time = 30 min ± 10 | | | |
| | EQ-036) | | min) | | | |

A non-limiting example of the mixing characteristics (plate's speed, inclination angle and phase of x- and y-axis) of the configured modes is shown in Table 9 below. Table 9 shows the steps configured for a rolling circle amplification step.

TABLE 9
1 L bag with 445 mL reaction volume

| x-axis | | | y-axis | | |
|---|---|---|---|---|---|
| Speed | | | Speed | | |
| (rpm) | Angle (°) | Phase (°) | (rpm) | Angle (°) | Phase (°) |
| 16 | 20 | 90 | 16 | 12 | 0 |
| 16 | 20 | 0 | 16 | 12 | 90 |
| 48 | 12 | 180 | 48 | 10 | 0 |
| 48 | 12 | 45 | 48 | 10 | 45 |
| 0 | 0 | 0 | 0 | 0 | 0 |

Agitation program at different volume of reactions, for example 445 ml, and 4450 ml.

During the enzymatic phases carried out in the inventive bioreactor for clDNA or neDNA (e.g., dbDNA) production process, in both scales (445 mL and 4450 mL), the same incubation-stirring programs were used. The purpose of the programmed agitations for both scales is to achieve an adequate mixing of the components during the different stages of the dbDNA production. The bag volumes used for 445 ml and 4450 ml reactions are different as shown in Table 10 below

TABLE 10

| | Used in 445 mL scale | Used in 4450 mL scale |
|---|---|---|
| Bag volume | 1 L | 10 L |
| Length [mm] | 299 | 621 |
| Width [mm] | 155 | 300 |
| Film surface area | 707 | 3528 |
| [mm²] | | |
| Characteristic | 1414.43 | 2834.47 |
| length, D [mm] | | |

The mixing efficiency to be achieved for a large scale (4450 mL) programmed at the same agitation will be higher, and therefore will mix equally or more efficient than compared to a small scale (445 mL).

### Example 8: Heating From Room Temperature to 30.0° C.

The time required for a 1 L and a 10 L Flexboy® bags, filled with 445 mL and 4450 ml of water, respectively, to be heated moveable plate of the inventive reactor was tested. These volumes corresponded with current production processes of clDNA. The water was heated from room temperature to 30.0° C. on the moveable plate of the inventive reactor. Example 8 was carried out without applying any agitation to the Flexboy® bags. The moveable plate and chamber air setpoints of the inventive reactor were configured at 30.0° C. The respective bags were placed on the moveable plate, which was at approximately 30.0° C.

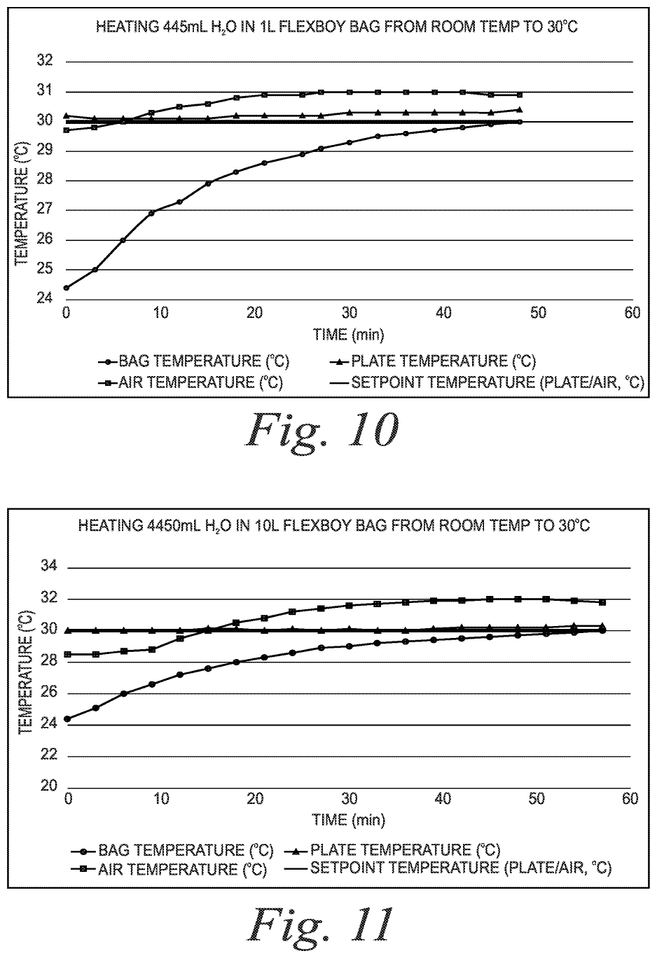

It took 48 minutes to heat the 445 mL of water in the 1 L Flexboy® bag from 24.4° C. to 30.0° C. (0.117° C./min). At 18 minutes, the solution was already within the incubation temperature acceptance range (set point±2.0° C.), exactly at 28.3° C. (0.094° C./min). See Table 11 below with the various temperature readings and FIG. 10 graphing the various temperatures during the heating of the 445 ml of water from room temperature to 30.0° C. using the inventive reactor in Example 8.

TABLE 11
Temperature Data Of 445 mL Of Water In 1 L Flexboy ® Bag

| | | Inventive Reactor | | |
|---|---|---|---|---|
| | Bag | Plate | Air | Setpoint |
| Time | temperature | temperature | Temperature | temperature |
| (min) | (° C.) | (° C.) | (° C.) | (Plate/Air, ° C.) |
| 0 | 24.4 | 30.2 | 29.7 | 30.0 |
| 3 | 25.0 | 30.1 | 29.8 | 30.0 |
| 6 | 26.0 | 30.1 | 30.0 | 30.0 |
| 9 | 26.9 | 30.1 | 30.3 | 30.0 |
| 12 | 27.3 | 30.1 | 30.5 | 30.0 |
| 15 | 27.9 | 30.1 | 30.6 | 30.0 |
| 18 | 28.3 | 30.2 | 30.8 | 30.0 |
| 21 | 28.6 | 30.2 | 30.9 | 30.0 |
| 25 | 28.9 | 30.2 | 30.9 | 30.0 |
| 27 | 29.1 | 30.2 | 31.0 | 30.0 |
| 30 | 29.3 | 30.3 | 31.0 | 30.0 |
| 33 | 29.5 | 30.3 | 31.0 | 30.0 |
| 36 | 29.6 | 30.3 | 31.0 | 30.0 |
| 39 | 29.7 | 30.3 | 31.0 | 30.0 |
| 42 | 29.8 | 30.3 | 31.0 | 30.0 |
| 45 | 29.9 | 30.3 | 30.9 | 30.0 |
| 48 | 30.0 | 30.4 | 30.9 | 30.0 |

The 4450 mL of water in the 10 L bag on the moveable plate of the inventive reactor required 57 minutes to reach 30.0° C. from 24.4° C. (0.098° C./min) and 18 minutes to reach the incubation acceptance temperature range (set point±2.0° C.) (0.2° C./min). See Table 12 below with the various temperature readings and FIG. 11 graphing the various temperatures during the heating of the 4450 ml of water from room temperature to 30.0° C. using the inventive reactor in Example 8.

TABLE 12
Temperature Data Of 4450 ml Of Water In 10 L Flexboy ® Bag

| | | Inventive Reactor | | |
|---|---|---|---|---|
| | Bag | Plate | Air | Setpoint |
| Time | temperature | temperature | Temperature | temperature |
| (min) | (° C.) | (° C.) | (° C.) | (Plate/Air, ° C.) |
| 0 | 24.4 | 30.0 | 28.5 | 30.0 |
| 3 | 25.1 | 30.0 | 28.5 | 30.0 |
| 6 | 26.0 | 30.0 | 28.7 | 30.0 |
| 9 | 26.6 | 30.0 | 28.8 | 30.0 |
| 12 | 27.2 | 30.0 | 29.5 | 30.0 |
| 15 | 27.6 | 30.1 | 30.0 | 30.0 |
| 18 | 28.0 | 30.1 | 30.5 | 30.0 |
| 21 | 28.3 | 30.0 | 30.8 | 30.0 |
| 24 | 28.6 | 30.1 | 31.2 | 30.0 |
| 27 | 28.9 | 30.0 | 31.4 | 30.0 |
| 30 | 29.0 | 30.1 | 31.6 | 30.0 |
| 33 | 29.2 | 30.0 | 31.7 | 30.0 |
| 36 | 29.3 | 30.0 | 31.8 | 30.0 |
| 39 | 29.4 | 30.1 | 31.9 | 30.0 |
| 42 | 29.5 | 30.2 | 31.9 | 30.0 |
| 45 | 29.6 | 30.2 | 32.0 | 30.0 |
| 48 | 29.7 | 30.2 | 32.0 | 30.0 |
| 51 | 29.8 | 30.2 | 32.0 | 30.0 |
| 54 | 29.9 | 30.3 | 31.9 | 30.0 |
| 57 | 30.0 | 30.3 | 31.8 | 30.0 |

### Example 9: Heating And Cooling Using 4450 mL Between 30.0° C. And 50.0° C.

The time required for a Flexboy® 10 L bag filled with 4450 ml of water to reach 50.0° C. from 30.0° C. was tested. The time required for the bag to cool from 50.0° C. to 30.0° C. on the moveable plate of the inventive reactor was also tested. During the testing, a slight agitation was applied to homogenize the temperature of the bag.

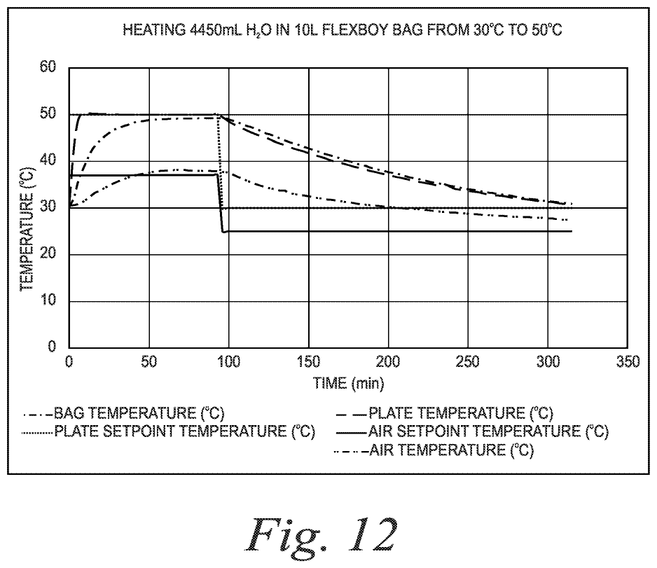

It took 72 minutes to heat 4450 ml of water from 30.2° C. to 49.2° C. (0.263° C./min), while 222 minutes were needed to cool down the water from 49.2° C. to 30.9° C. (−0.082° C./min). However, during the heating step at 39 minutes, the solution was already within the incubation temperature acceptance range (set point±2.0° C.), exactly at 48.1° C. (0.094° C./min). During the cooling-down phase, 195 minutes were needed to reach the incubation temperature acceptance range (−0.088° C./min). See FIG. 12 graphing the various temperatures during the heating and cooling of Example 9.

The rate at which the solution in bags was heated in the inventive reactor varied based on the volume in the bag, in addition to the power that the stirrer-movable plate used to maintain the set temperature setpoint. The larger the volume to be heated, the longer it took to reach the temperature setpoint set on the moveable plate. The greater the temperature difference between the initial and final temperatures of the moveable plate, the greater the power used by the inventive reactor to heat the moveable plate and, consequently, the greater the heating rate of the solution in a given bag for the same volume. The cooling rate for the same volume of solution and the same difference in initial and final temperature of the moveable plate was lower than in heating.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

1. A reactor comprising:
a support base;
at least one wall;
a top cover, wherein the top cover, the support base and the at least one wall form an internal chamber therein; and
a moveable plate assembly including a moveable plate located within the internal chamber, the moveable plate being configured to move in the x-direction, y-direction, and the z-direction, the temperature of the moveable plate being controllable to a desired temperature,
wherein the internal chamber is accessible via at least one of the top cover, the support base, and the at least one wall.

2. The reactor of claim 1, wherein the top cover, the support base, and the at least one wall comprise stainless steel.

3. The reactor of claim 1, wherein the at least one wall forms a porthole for viewing into the internal chamber.

4. The reactor of claim 3, wherein the at least one wall is a plurality of walls, the plurality of walls forming a respective porthole for viewing into the internal chamber.

5. The reactor of claim 1, wherein the internal chamber is accessible by moving a portion of the at least one wall and a portion of the top cover via a hinge.

6. The reactor of claim 1, wherein the moveable plate comprises aluminum.

7. The reactor of claim 1, wherein the moveable plate comprises a plurality of layers.

8. The reactor of claim 7, wherein the moveable plate comprises a resistive heating element layer.

9. The reactor of claim 1, wherein the maximum inclination of the moveable plate is about 20 degrees in any direction.

10. The reactor of claim 9, wherein the maximum inclination of the moveable plate is about 30 degrees in any direction.

11. The reactor of claim 1, wherein the movement of the moveable plate uses linear movement, rotary movement and orbital movement.

12. The reactor of claim 1, wherein the reactor has a width of from about 500 mm to about 1,500 mm, a height of from about 500 mm to about 2,000 mm, and a depth of from about 500 mm to about 1,500 mm.

13. The reactor of claim 1 further including filters to assist in preventing or inhibiting cross-contamination.

14. The reactor of claim 1, wherein the at least one wall is a plurality of walls.

15. The reactor of claim 1, wherein the internal chamber is hermetically closed.

16. A method of using a reactor for an enzymatic reaction, the method comprising:
providing a reactor, the reactor including a support base, at least one wall, a top cover, and a moveable plate, the top cover, the support base and the at least one wall form an internal chamber therein, the moveable plate assembly including a moveable plate located within the internal chamber, the moveable plate being configured to move in the x-direction, y-direction, and the z-direction, the temperature of the moveable plate being controllable to a desired temperature, the internal chamber being accessible via at least one of the top cover, the support base, and the at least one wall;
placing a bag or package on the moveable plate in a secured position, the bag including at least one reaction component and an enzyme;
heating the moveable plate to a desired temperature;
controlling the temperature within the internal chamber; and
moving the moveable plate in the x-direction, y-direction and the z-direction so as to mix the at least one reaction component and the enzyme.

17. The method of claim 16, wherein the bag is from about 1 to about 20 liters.

18. The method of claim 16, wherein the at least one reaction component is a nucleic acid.

19. The method of claim 18, wherein the nucleic acid is DNA and/or RNA.

20. The method of claim 16, wherein the enzyme is restriction endonuclease; ligase; protelomerase; endonuclease; exonuclease; Phi 29 DNA polymerase; DNA polymerase or any variant thereof; reverse transcriptase; DNA recombinase; Cre recombinase; protease; nuclease; Taq DNA polymerase; phosphorylase; phosphotransferase; dephosphorylase; nucleic acid synthase; RNA polymerase; meganucleases; zinc finger nucleases (ZFNs); transcription activator-like effector-based nucleases (TALEN); or Cas9 nuclease.

21. The method of claim 16, wherein the at least one reaction component is RNA, DNA, plasmid DNA, deoxynucleoside triphosphate (dNTPs), oligonucleotides, inhibitory nucleic acids, gRNA, modified DNA, or any combination thereof.

22. The method of claim 16, wherein the enzyme is involved in an enzymatic reaction, the enzymatic reaction being restriction digestion with restriction endonucleases; nucleic acid ligation; reaction with protelomerase; reaction with endonuclease and/or exonuclease; reaction with DNA polymerase or any variant thereof; reaction with reverse transcriptase; reaction with recombinase; reaction with protease; reaction with nuclease; polymerase chain reaction; nucleic acid amplification reaction; phosphorylation reaction; dephosphorylation reaction; nucleic acid synthesis reaction; reaction with RNA polymerase; reaction of nick or damage repair of nucleic acid; nucleic acid denaturation reaction; nucleic acid annealing reaction; or nucleic acid extension reaction.
